(19) United States
(12) Patent Application Publication
(10) Pub. No.: US 2018/0145443 A1
(43) Pub. Date: May 24, 2018

(54) CONNECTOR AND METHODS FOR MAKING AND USING THE CONNECTOR

(71) Applicant: Lucent Medical Systems, Inc., Kirkland, WA (US)

(72) Inventors: Samuel Peter Andreason, Kirkland, WA (US); Steve Vincent, Kirkland, WA (US); Curtis S. King, Kirkland, WA (US)

(21) Appl. No.: 15/820,209

(22) Filed: Nov. 21, 2017

Related U.S. Application Data
(60) Provisional application No. 62/425,004, filed on Nov. 21, 2016.
(60) Provisional application No. 62/425,002, filed on Nov. 21, 2016.

(51) Int. Cl. H01R 13/52; H01R 24/40; H01R 13/04; H01R 13/11; H01R 43/26; H01R 43/18; A61B 5/06; A61B 34/20; A61B 46/17

(52) U.S. Cl. CPC H01R 13/5224; H01R 24/40; H01R 13/04; H01R 13/111; H01R 43/26; A61B 2560/0487; A61B 5/062; A61B 34/20; A61B 46/17; H01R 2201/12; A61B 2034/2051; H01R 43/18

(57) ABSTRACT

A multipart connector is employed in a system that tracks a medical device in the human body. The multipart connector includes a first connector portion and a second connector portion wherein the first connector portion pierces a contamination barrier to couple with the second connector portion. The medical device is a trackable structure having an integrated electromagnet circuit. The trackable structure is arranged to controllably produce a magnetic field. An interface is provided in the system to produce a positional representation of the trackable structure when the trackable structure is placed and moved within the human body. A magnetic field sensing device drives the integrated electromagnet circuit of the trackable structure tracks medical device and provides position information to the interface. The multipart connector electrically couples the magnetic field sensing device to the trackable structure.

### CROSS-REFERENCE(S) TO RELATED APPLICATION(S)

This application claims the benefit of U.S. Provisional Patent Application No. 62/425,002, filed Nov. 21, 2016, and U.S. Provisional Patent Application No. 62/425,004, filed Nov. 21, 2016, both of which are hereby incorporated by reference in their entirety to the extent that they do not conflict with the present specification.

### BACKGROUND

### Technical Field

The present disclosure generally relates to an electrical connector associated with an electromagnetic tracking device that is movable within a body of a patient. More particularly, but not exclusively, the present disclosure relates to a multipart electrical connector system used in a medical environment wherein a first portion of the connector system is arranged to pierce a malleable barrier in cooperation with coupling the first portion of the connector system to a second portion of the connector system.

### Description of the Related Art

In many medical procedures, a medical practitioner accesses an internal cavity of a patient using a medical instrument. In some cases, the medical practitioner accesses the internal cavity for diagnostic purposes. In other cases, the practitioner accesses the cavity to provide treatment. In still other cases different therapy is provided.

Due to the sensitivity of internal tissues of a patient's body, incorrectly positioning the medical instrument within the body can cause great harm. Accordingly, it is beneficial to be able to precisely track the position of the medical instrument within the patient's body. However, accurately tracking the position of the medical instrument within the body can be difficult. The difficulties are amplified when the medical instrument is placed deep within the body of a large patient.

In many hospitals, a medical practitioner uses electrical connectors while concurrently using various medical instruments. In some cases, the medical practitioner uses medical devices having one or more electrical connectors for diagnostic purposes and for administering medication. In other cases, the practitioner uses several electrical connectors and devices to monitor a patient's vital signs. In still other cases different electrical connectors are provided.

In many circumstances, a medical practitioner uses electrical connectors in cooperation with electrical devices that monitor a patient's vital signs while a medical procedure is performed. In some cases, the medical practitioner uses electrical connectors with one or more monitors that collect information associated with the patient's heartbeat, temperature, and other vital signs. In addition, the medical practitioner may use electrical connectors to facilitate the operation of devices that administer medication during the medical procedure. In still other cases different electrical connectors are provided and used for other purposes.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### BRIEF SUMMARY

Electrical connector systems and methods are arranged to couple one or more low-frequency electromagnetic trackable structures to magnetic field sensing devices are described, and systems and methods to form such electrical connectors are described.

A first embodiment of a system may be summarized as including a trackable structure having an integrated electromagnet circuit. The trackable structure is arranged to controllably produce a magnetic field. The system also includes an interface to produce a positional representation of the trackable structure when the trackable structure is within a human body, a magnetic field sensing device arranged to drive the integrated electromagnet circuit of the trackable structure and arranged to provide position information to the interface, and a multipart connector to electrically couple the magnetic field sensing device to the trackable structure. The multipart connector including a first connector portion and a second connector portion.

The trackable structure of the first embodiment may further include a medical device and a trackable conductor. In some of these cases, the trackable conductor is arranged to receive an electromagnetic drive signal and arranged to generate an electromagnetic field in correspondence with the electromagnetic drive signal. What's more, in some of these cases, the magnetic field sensing device is arranged to generate position information representing a location of the trackable structure in real time by sensing the electromagnetic field generated by the trackable conductor.

In some other cases, the first connector portion and the second connector portion of the first embodiment are arranged to form at least one electrically conductive path through the multipart connector when the first connector portion and the second connector portion are mechanically joined together. Sometimes, the magnetic field sensing device is configured to direct passage of an electromagnetic drive signal through the at least one electrically conductive path. And sometimes, the first connector portion includes an electrically conductive core having a body, a distal end, and a core electrical contact area formed on the distal end of the electrically conductive core; a first insulator layer substantially surrounding the body of the electrically conductive core; a first conductive shield layer substantially surrounding the first insulator layer, the first conductive shield layer having a body, a distal end, and a first electrical contact area formed on the distal end of the first conductive shield layer; a second insulator layer substantially surrounding the body of the first conductive shield layer; a second conductive shield layer substantially surrounding the second insulator layer, the second conductive shield layer having a body, a distal end, and a second electrical contact area formed on the distal end of the second conductive shield layer; and a third insulator layer substantially surrounding the body of the second conductive shield layer. In some of these cases, the core electrical contact area, the first electrical contact area, and the second electrical contact area are exposed to an outside environment. In some of these cases, the distal end of the electrically conductive core, the distal end of the first conductive shield layer, and the distal end of the second conductive shield layer are configured to pass through a contamination barrier when the first connector portion and the second connector portion are mechanically joined together. In some of these cases, the second connector includes an electrically conductive conduit having a body, a distal end, and an electrical receiver arranged to receive the core electrical contact area of the first connector portion; a third insulator layer substantially surrounding the body of the electrically conductive conduit; a third conductive shield layer substantially surrounding the third insulator layer, the third conductive shield layer having a body, a distal end, and a third electrical receiver formed at the distal end of the third conductive shield layer, the third electrical receiver arranged to receive the first electrical contact area; a fourth insulator layer substantially surrounding the body of the third conductive shield layer; a fourth conductive shield layer substantially surrounding the fourth insulator layer, the fourth conductive shield layer having a body, a distal end, and a fourth electrical receiver formed at the distal end of the fourth conductive shield layer, the fourth electrical receiver arranged to receive the second electrical contact area; and a fifth insulator layer substantially surrounding the body of the fourth conductive shield layer. And in some of these cases, the first connector portion includes a shroud arranged to at least partially enclose the core electrical contact area, the first electrical contact area, and the second electrical contact area.

A method embodiment may be summarized as including providing a contamination barrier to separate a first space from a second space; providing a first connector portion of a multipart connector in the first space, wherein the first connector portion is arranged for coupling to a magnetic field sensing device; providing a second connector portion of the multipart connector in the second space, wherein the second connector portion is arranged for coupling to a trackable structure having an integrated electromagnet circuit, the trackable structure arranged to controllably produce a magnetic field; passing at least one electrical conductor of the first connector portion through the contamination barrier; and mechanically coupling the first connector portion to the second connector portion thereby forming at least one electrically conductive path through the contamination barrier.

In some cases of this method, the first space has a first level of sterility and the second space has a second level of sterility, the first level of sterility representing a less sterile condition than the second level of sterility. In some cases, the method also includes, prior to passing the at least one electrical conductor of the first connector portion through the contamination barrier, and prior to mechanically coupling the first connector portion to the second connector portion, coupling the second connector portion to the trackable structure. And in some cases, the method includes applying an electromagnetic drive signal to the integrated electromagnet circuit via the at least one electrically conductive path. In some of these cases, applying the electromagnetic drive signal further comprises passing an alternating current excitation signal through the at least one electrically conductive path to the integrated electromagnet circuit of the trackable structure, the alternating current excitation signal having a frequency below 10,000 Hz.

And yet another method embodiment is a method to form a first electrical connector. This method may be summarized as including providing an electrically conductive core having a distal end and a body; forming a first insulator layer substantially surrounding the body of the electrically conductive core, the first insulator layer formed substantially coaxial with the electrically conductive core; exposing a core electrical contact area on the distal end of the electrically conductive core; forming a first conductive shield layer substantially surrounding the first insulator layer, the first conductive shield layer having a body and a distal end, the first conductive shield layer formed substantially coaxial with the first insulator layer; forming a second insulator layer substantially surrounding the first conductive shield layer, the second insulator layer formed substantially coaxial with the first conductive shield layer; exposing a first electrical contact area on the distal end of the first conductive shield layer; forming a second conductive shield layer substantially surrounding the second insulator layer, the second conductive shield layer having a body and a distal end, the second conductive shield layer formed substantially coaxial with the second insulator layer; forming a third insulator layer substantially surrounding the second conductive shield layer, the third insulator layer formed substantially coaxial with the second conductive shield layer; and exposing a second electrical contact area on the distal end of the second conductive shield layer. In some cases of the method to form a first electrical connector, the distal end of the electrically conductive core is arranged to pierce a contamination barrier.

One more method is a method to form a second electrical connector. Embodiments of this method include providing an electrically conductive multi-leaf receiver having a first electrical receiver end and a body; forming a first insulator layer substantially surrounding the body of the electrically conductive multi-leaf receiver, the first insulator layer formed substantially coaxial with the electrically conductive multi-leaf receiver; forming a first electrically conductive receiver substantially surrounding the first insulator layer, the first electrically conductive receiver having a second electrical receiver end and a body, the first electrically conductive receiver substantially coaxial with the first insulator layer; forming a second insulator layer substantially surrounding the body of the first electrically conductive receiver, the second insulator layer formed substantially coaxial with the first electrically conductive receiver; forming a second electrically conductive receiver substantially surrounding the second insulator layer, the second electrically conductive receiver having a second electrical receiver end and a body, the second electrically conductive receiver substantially coaxial with the second insulator layer; and forming a third insulator layer substantially surrounding the body of the second electrically conductive receiver, the third insulator layer formed substantially coaxial with the second electrically conductive receiver. In some cases, the bodies of the insulator layers and the electrically conductive receivers are flexible.

Embodiments of a first electrical connector may be summarized as including a group of electrical connector pins arranged to pass through a contamination barrier and pass an electromagnetic drive signal, each electrical connector pin has a distal end; an electrically conductive path coupled to the group of electrical connector pins, the electrically conductive path is arranged to pass the electromagnetic drive signal; an electrical housing that contains the electrical connector pins and the electrically conductive path; and an insulating material inside the electrical housing, the insulating material holds the group of electrical connector pins and the electrically conductive path in place. In some cases, the distal ends of the group of electrical connector pins are arranged to pass through a contamination barrier.

Embodiments of a second electrical connector may be summarized as including a group of electrical pin receivers arranged to receive a first electrical connector and pass an electromagnetic drive signal, each electrical pin receiver has an electrical receiver end; an electrically conductive path coupled to the group of electrical pin receivers, the electrically conductive path is arranged to pass the electromagnetic drive signal; an electrical housing that contains the electrical pin receivers and the electrically conductive path; and an insulating material located inside the electrical housing, the insulating material holds the electrical pin receivers and the electrically conductive path in place. In some cases, the electrical receiver ends of the group of electrical pin receivers are arranged to receive a group of electrical connector pins.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. The shapes of various elements and angles are not necessarily drawn to scale either, and some of these elements are enlarged and positioned to improve drawing legibility. One or more embodiments are described hereinafter with reference to the accompanying drawings in which:

FIG. 1 illustrates a medical procedure embodiment in which an electrical connector system embodiment is implemented;

FIG. 2 is a first electrical connector embodiment and a second electrical connector embodiment of a medical electrical connector system embodiment;

FIG. 3 is a first electrical connector embodiment and a second electrical connector embodiment that includes an ECG connector embodiment of the medical electrical connector system embodiment;

FIG. 4 is a method to produce one embodiment of the first electrical connector of the medical electrical connector system embodiment;

FIG. 5 is a first electrical connector embodiment;

FIG. 6 is a trackable structure embodiment;

FIG. 7 is a first electrical connector embodiment;

FIGS. 8A-8C are shroud embodiments of the first electrical connector;

FIG. 9 is a second electrical connector embodiment;

FIG. 10 is a multipart connector to electrically couple a magnetic field sensing device to the trackable structure;

FIGS. 11A-11B are first and second electrical connector embodiments, respectively, that cooperate in a connector system such as the medical electrical connector system embodiment of FIG. 2;

FIGS. 11C-11D are cross-sections of the first and second electrical connector embodiments of FIGS. 11A-11B, respectively;

FIG. 11E illustrates the connector embodiment of FIG. 11A passing through a contamination barrier;

FIG. 11F illustrates another embodiment of the second electrical connector of FIG. 11B;

FIG. 11G illustrates a portion of a cooperative coupling method between a first electrical connector of FIG. 11A and a second electrical connector of FIG. 11F;

FIGS. 12A-12D are piercing structure embodiments;

FIGS. 13A-13D are optional piercing structure sharpened edge embodiments;

FIG. 14 is a two-stage connector housing embodiment viewed from a first perspective;

FIG. 15 is the two-stage connector housing embodiment of FIG. 14 viewed from a second perspective;

FIG. 16 is the two-stage connector housing embodiment of FIG. 14 with partial installation of an electrical contact/cable assembly;

FIG. 17A is a sectional view of the two-stage connector housing embodiment of FIG. 14 with partial installation of the electrical contact/cable assembly from a top view perspective;

FIG. 17B is a detail view of a portion of the two-stage connector housing embodiment of FIG. 17A from a side view perspective;

FIG. 18 is a front view of the two-stage connector housing embodiment;

FIG. 19 is a two-stage connector receiver embodiment beneath an exemplary contamination barrier;

FIG. 20A is a two-stage connector housing embodiment and a two-stage connector receiver embodiment aligned for electromechanical coupling through a contamination barrier;

FIG. 20B is a detail view of a portion of the two-stage connector housing and two-stage connector receiver aligned for electromechanical coupling through a contamination barrier;

FIG. 21A is a two-stage connector housing embodiment and a two-stage connector receiver embodiment aligned for direct electromechanical coupling;

FIG. 21B is a detail view of a portion of the two-stage connector housing and two-stage connector receiver aligned for direct electromechanical coupling;

FIG. 22 is a sectional view of the two-stage connector housing and two-stage connector receiver coupled through a contamination barrier;

FIG. 23A shows a two-stage connector housing embodiment in an open position;

FIG. 23B shows the two-stage connector housing embodiment of FIG. 23A advanced to a closed position;

FIG. 24A is a two-stage connector housing embodiment in a closed and locked position;

FIG. 24B is a detail view of the portion of the two-stage connector housing;

FIG. 25A is a sectional view of the two-stage connector housing embodiment from a top view perspective;

FIG. 25B is a detail view of a portion of the two-stage connector housing embodiment of FIG. 25A from a side view perspective; and

FIG. 25C is a more detailed view of the portion of the two-stage connector housing embodiment of FIG. 25B.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with computing systems including client and server computing systems, as well as networks have not been shown or described in detail to avoid unnecessarily obscuring descriptions of the embodiments.

A medical device having a new electromechanical connector structure is contemplated. The electromechanical connector structure includes a first connector apparatus that is capable of passing through (e.g., piercing) a contamination barrier and a second connector apparatus that is configured to receive the first connector.

The term, “contamination barrier,” as used herein, may interchangeably be referred to as a surgical drape, a drape surgical sheet, a surgical sheet, a draw pad sheet, an operation theater sheet, an incision film, scrubs, or the like. The contamination barrier may be formed in any shape such as a rectangle, and generally, the contamination barrier is flexible. The contamination barrier may be formed from one or more non-woven materials, fibrous materials, or other materials, which may be arranged, for example, in layers. One or more layers may be resistant to liquids or even impermeable by liquids such as bodily fluids. One or more layers may be highly absorbent. The contamination barrier is generally sterilized and packaged at the time of manufacture to maintain sterility until the time of use in a medical procedure. The contamination barrier may be arranged from tear-resistant materials or formed in a tear-resistant way. The contamination barrier may form a barrier to contaminants, or provide some other benefit during a medical procedure.

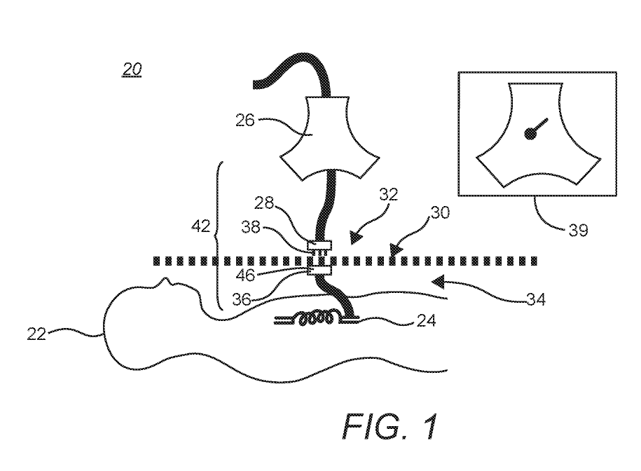

FIG. 1 illustrates a medical procedure embodiment in which an electrical connector system 20 embodiment is implemented. A patient 22 is undergoing a medical procedure. The patient 22 may be a human patient or a non-human patient. The medical electrical connector system 20 in one embodiment, generally, comprises a first electrical connector 28, a second electrical connector 36, a magnetic field sensing device 26, and a trackable structure 24.

A medical practitioner (not shown) is administering the procedure. The medical practitioner is directing movement of the trackable structure 24 within the body of the patient 22. The trackable structure 24 may be a stylet, a catheter such as a Peripherally Inserted Central Catheter (PICC), a medical tube, a tracheal tube, a needle, a cannula, or some other structure. In some cases, the trackable structure 24 is a hollow, tube-like device. In some cases, the trackable structure 24 is an elongated, solid member. In some cases, the trackable structure 24 takes another form.

The medical electrical connector system 20 disclosed herein allows the medical practitioner to form an electrically conductive path 42 through a contamination barrier 30 to pass signals (e.g., power, control, sense, and the like) to the trackable structure 24, from the trackable structure 24, or to and from the trackable structure 24. The term, electrically conductive path 42, as used in the present disclosure may include one electrical conductive conduit or a plurality of electrically conductive conduits.

The medical practitioner uses the first electrical connector 28 or another suitable device in the first space 32 to pass through (e.g., pierce, slice, cut, penetrate) a contamination barrier 30 into a second space 34. The first electrical connector 28 is coupled to the medical sensing device 26.

The contamination barrier 30, or some other structure, may impede the view of the trackable structure 24, the second electrical connector 36, or other structures in the second space 34 during the medical procedure.

In the embodiment of FIG. 1, after the contamination barrier is pierced, the first electrical connector 28 is electromechanically coupled to the second electrical connector 36. The coupling may be in a direct electrical connection or the coupling may be through one or more intervening devices. The coupling may also include a mating or other association of one or more mechanical registration features integrated with the first electrical connector 28, the second electrical connector 36, or both the first electrical connector 28 and the second electrical connector 36. Furthermore, coupling the first electrical connector 28 to the second electrical connector 36 involves coupling a group of one or more electrical connector (i.e., electrically conductive) pins 38 to a group of one or more electrical connector (i.e., electrically conductive) pin receivers 46. The group of electrical connector pins 38 being removably or fixedly integrated with the first electrical connector 28. The group of electrical connector pin receivers 46 being removably or fixedly integrated with the second electrical connector 36. By coupling the first electrical connector 28 to the second electrical connector 36, the medical practitioner forms an electrically conductive path 42.

In some embodiments, before and after the coupling, the combination of first electrical connector 28 and the second electrical connector 36 may be referred to as a multipart connector having a first electrical connector portion and a second electrical connector portion. In some embodiments described herein, rather than the first electrical connector 28 piercing the contamination barrier, the second electrical connector 36 pierces the contamination barrier. That is, the direction from which the contamination barrier is pierced may be from an outside space, an inside space, an above patient space, a below patient space, an above barrier space, a below barrier space, or from some other space. In pursuit of brevity, not every contemplated arrangement or direction in which the connector passes through the contamination barrier is described.

As described herein, the first space 32 may be an outside space of the contamination barrier, an inside space of the contamination barrier, an unsterile region, an unsterile space, an unsterile area, an unsterile volume, or some other space altogether.

The second space 34 may be an inside space of the contamination barrier, an outside space of the contamination barrier, a sterile region, a sterile space, a sterile area, a sterile volume, or some other space altogether. The second electrical connector 36 is placed in the second space 34 before coupling to the first electrical connector 28. In some embodiments, the second electrical connector 36 and the trackable structure 24 are placed in the second space 34 before the medical practitioner begins the medical procedure. The second electrical connector 36 is coupled to the trackable structure 24. In some embodiments, the first space 32 has a first level of sterility, the second space 34 has a second level of sterility, and the first level of sterility represents a less sterile condition than the second level of sterility.

By using some portion or all of the first electrical connector 28 to pass through the contamination barrier 30, the medical practitioner has no need to move or lift the contamination barrier 30 to form the electrically conductive path 42. Thus, by utilizing the first electrical connector 28 to pass through a contamination barrier and by placing a second electrical connector 36 inside the contamination barrier before the medical procedure begins, the chance of a possible exposure of a sterile space to possible sources of contamination is reduced.

In the medical procedure embodiment of FIG. 1, after the electrically conductive path 42 is formed, the magnetic field sensing device 26 directs the passage of an electromagnetic drive signal through the electrically conductive path 42 formed via the first and second electrical connectors 28, 36 to the trackable structure 24. The electromagnetic drive signal may include one or more of power, control, data, and the like. In some embodiments, the electromagnetic drive signal is an alternating current excitation signal having a frequency below 10,000 Hz. In some embodiments, the electromagnetic drive signal is an alternating current excitation signal having a frequency below 1,000 Hz. And in some embodiments, the electromagnetic drive signal is an alternating current excitation signal having a frequency below 500 Hz. In reliance on the receipt of power and suitable control signal information, the trackable structure 24 generates an electromagnetic field, which may be sensed by the magnetic field sensing device 26. In this way, the medical practitioner then uses the magnetic field sensing device 26 to track the powered trackable structure 24 as it is placed, moved, or otherwise passed in the body of a patient.

As represented in the embodiment of FIG. 1, the magnetic field sensing device 26 is communicatively coupled to an interface and display system 39. Using the interface and display system 39, the medical practitioner may easily determine the position (i.e., location), orientation, and optionally, one or more other information datums associated with the trackable structure 24 in real time.

Information that includes or is otherwise used to generate the position information passed to the interface and display system 39 is passed from the magnetic field sensing device 26. The magnetic field sensing device 26 is coupled to a first portion of the electrically conductive path 42. The first portion of the electrically conductive path 42 is coupled to the first electrical connector 28. In addition, the trackable structure 24 is coupled to a second portion of the electrically conductive path 42. The second portion of the electrically conductive path 42 is coupled to the second electrical connector 36.

The trackable structure 24 may enter the body through the mouth of the patient 22 or through another of the patient's orifices. Alternatively, the trackable structure 24 may be placed or otherwise passed through a surgical incision made by the same medical practitioner or a different medical practitioner at some location on the body of the patient 22. The trackable structure 24 may be placed in other ways.

The magnetic field sensing device 26 is operated by a medical practitioner proximal to the body of the patient 22. In some cases, the medical practitioner places the magnetic field sensing device 26 directly in contact with the body of the patient 22. In other cases, the magnetic field sensing device 26 is operated in proximity to the body of the patient 22 without directly contacting the body of patient 22. In many cases, the medical practitioner will attempt to place the magnetic field sensing device 26 adjacent to the portion of the body where the trackable structure 24 is believed to be.

To improve the results in medical procedures that employ trackable structures 24 and medical sensing devices 26, stray electromagnetic fields from the leads (e.g., supply wires) that drive coils formed on the trackable structures 24 are desirably controlled to prevent the introduction of an artificial ‘offset’ signal into captured magnetic sensor data. That is, to improve performance of the tracking system, the drive fields are preferably confined to the drive leads as much as possible.

In some cases, a zeroing calibration step can also limit the impact of stray fields. Generally speaking, however, the zeroing calibration step performs better when the nature of the drive signals is repetitive and not fluctuating over long time scales. The zeroing calibration step may be undesirable for at least two reasons. First, the transmitting coil of a trackable structure 24 may in some cases need to be placed far enough away from the medical sensing devices 26 so as to present a negligible signal to the magnetic sensors of the medical sensing devices 26 during the time the zeroing step is performed. This action may be burdensome in practice, however, because the distances can be quite large. Second, the transmitting coil's drive current characteristics may need to be sufficiently consistent such that a predictable, predetermined “factory” subtraction value can be sufficiently accurate to remove the impact of the stray fields. In these cases, it has been shown that with consistent manufacturing of transmitting coils and transmission lines across a plurality of production runs of trackable structures 24, a predetermined factory zeroing value may be acceptably determined within about +/−10% of accurate. Both of the two approaches may still be used even when efforts are made to lower the natural stray fields from the coils of trackable structures 24 as by the connector embodiments described herein. In yet some other cases, actual current waveforms may be digitized and processed to allow a factory subtraction value to be scaled for an actual trackable structure 24 (e.g., stylet) in use, but such procedures also add complications and the potential for additional coupling to the coil drive circuit.

Another mechanism to reduce stray electromagnetic fields includes the use of tightly twisted pairs of lead wires. The use of tightly twisted pairs may help lower stray fields from the electrical connections in general. Relative to the cost of an entire tracking system, twisted pair lead wires are inexpensive and effective, so many embodiments will employ them. On the hand, the nature of the twisted pair cannot easily be maintained through a connector if at all. That is, within the connector, conductive drive lines will typically run untwisted at least for some nominal length.

As described herein, for at least some medical applications, it is desirable for an electrical connector that couples drive wires associated with a medical sensing device 26 to a trackable structure 24 to be arranged to also pass through a contamination boundary. Conventionally, this has been accomplished with connectors having straight pins capable of penetrating a drape to make electrical contact.

To complete a circuit, two electrical connections are made, and the size of the loop area created with these two pins determines the level of external electromagnetic fields that are generated. In some cases, the pins are moved closer together, which results in less contamination. Notably, however, there is a mechanical limitation at least for alignment purposes as to how close the pins can be moved. In some other embodiments, there is also a desire to have the pins of the connector be as long as possible so that a wide variety of contamination barrier thicknesses can be accommodated.

Yet one more consideration relevant to at least some embodiments is a design configuration such that the barrier being pierced (e.g., contamination barrier 30) does not lose any pieces that either compromise the patient's medical procedure or get detrimentally deposited (e.g., pressed, forced, dragged) into the connector. This consideration introduces differences between standardized conventional coaxial connectors, standardized conventional BNC style connectors, and the inventive connector designs described herein.

In some embodiments, as described herein, connector pins are arranged in sets. For example, a center “outgoing signal” pin may be surrounded by multiple “incoming signal” return pins. This type of structure may include a planar geometry (i.e., three pins in a row) or in other embodiments, a centrally arranged pin is surrounded by some number (e.g., four) of signal pins: top, left, right, bottom). These arrangements may still leak electromagnetic fields into their surroundings, but less so than with two simple pins. More specifically, the magnetic fields from these arrangements will generally fall off faster with distance. More specifically still, the arrangements strive to eliminate the lower order terms of the magnetic field as a function of distance.

In at least one exemplary solution, a first connector is formed coaxial in nature such that the pin that pierces the contamination barrier 30 has an inner central portion and an outer cylindrical portion that is isolated by an insulator. In such embodiments, a leading tip (e.g., end, apex, crown, or the like) of the connector is formed with or having a point, a blade, or some other piercing (i.e., cutting, penetrating, boring, and the like) structure. Correspondingly, a second connector is formed as a receptacle portion that receives the first connector.

Exemplary embodiments of the receptacle portion may be formed with concentric sets of contact fingers. Conceptually, various embodiments may provide the first connector and theoretically increase the number of outer pins to infinity. In these embodiments, the center pin may part the barrier to either side. In this way, and based on the geometry of the coaxial connector, external magnetic fields may be substantially reduced or eliminated, which is readily understood in view of Ampere's law.

Considering Ampere's law, the path integral of magnetic field around a closed loop is proportional to the total current passing through the surface that the loop forms. As a coaxial connector is rotationally symmetric, and can be approximated as infinite in length, there can only be an azimuthal magnetic field. As the net current is zero, this azimuthal magnetic field must be zero. The diameter of the coaxially structured connector embodiments described herein can be significant. However, it is desirable that the center be concentric with respect to the outer conductive surface. This type of structure leads to a connector that can reliably find its mate while penetrating a contamination barrier 30.

In some embodiments, it is desirable to controllably maintain a uniform current distribution throughout the “shield” of the coaxial arrangement by, for example, carefully feeding currents into the connector. Lines that feed such currents may desirably be coaxially formed.

In some embodiments, additional low current electrical conduits are also desirable. In these cases, one or more additional barrier piercing pin(s) or layers may be added to the connector. In the case of an electrocardiograph (ECG) stylet, other options may also be considered. For example, so long as the contact resistance of the connector is sufficiently low, the ECG signal may be carried over through a pre-existing pin used by the stylet coil. This arrangement is potentially a more desirable system in that it would simplify the connector. On the other hand, such a connector may add complication to the design of the coil drive circuit. The complication may arise because the pre-existing pin may effectively become part of the ECG circuit and may impact such characteristics as the impedance matching of the ECG leads. One application where such complication may be noted is in a saline column type application.

The requirement in some embodiments of low contact resistance for a shared pin may be reduced by providing an operating frequency of the stylet (e.g., 330 Hz) that is greater than the bandwidth of the ECG system (e.g., 150 Hz). This assumed ECG system may not have sufficient bandwidth for pacemaker detection (300 Hz to 1 kHz). Given the clock-like nature of the coil drive circuit in the embodiments described herein, a bandstop filter (e.g., 330, 660, 990 Hz) may be implemented to allow ECG operation at higher frequencies. Some or all of these configurations may also have some impact on the detection of a “leads-off” condition, which system also operates at a higher frequency. In at least some embodiments, the contact resistance will fall somewhere in the range of 0.1 m-ohms to 10 m-ohms, and such values are consistent with being able to make a functioning ECG system, wherein ECG signal level is under 4 mV and peak coil currents are about 150 m-amps.

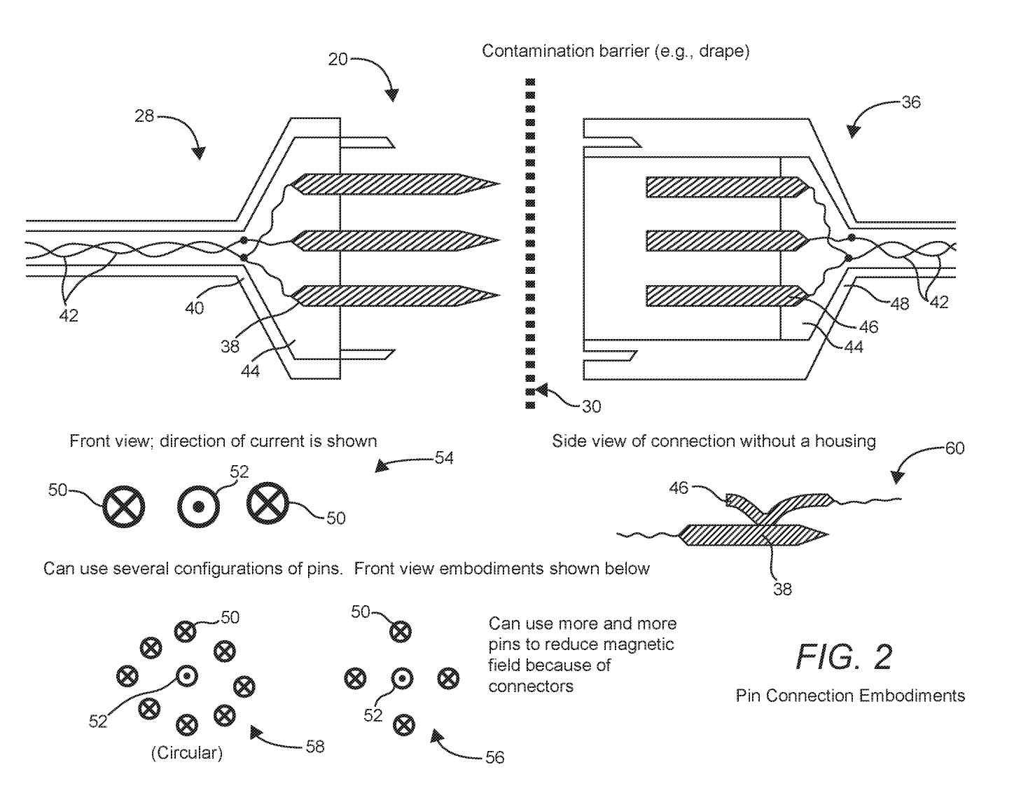

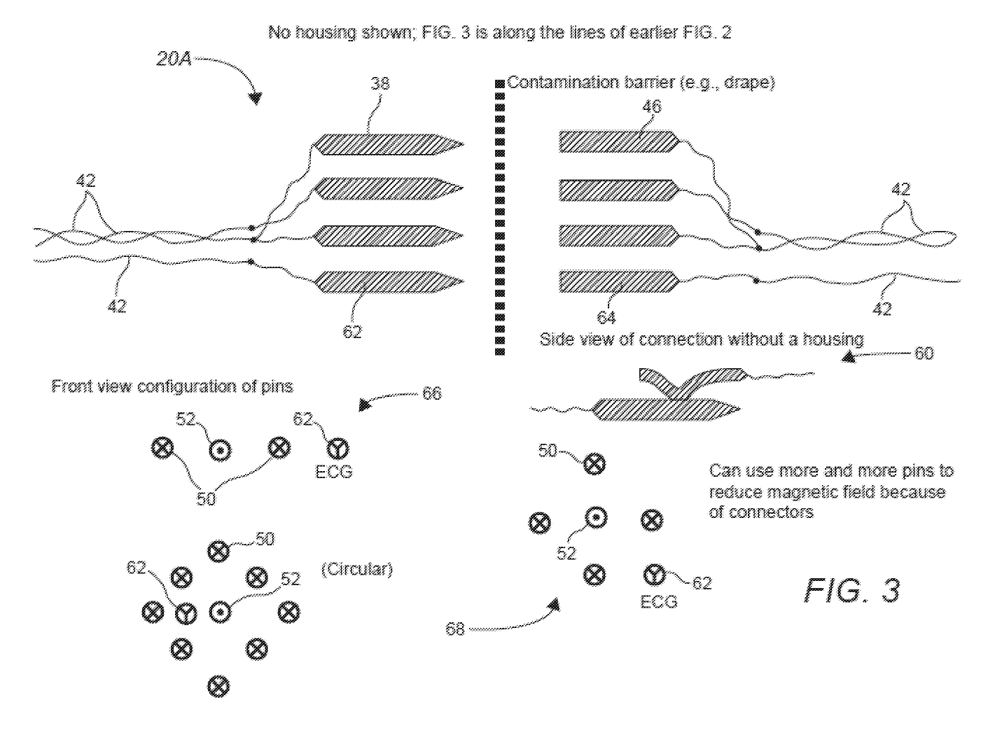

FIG. 2 is a first electrical connector 28 embodiment and a second electrical connector 36 embodiment of a medical electrical connector system 20 embodiment. The medical electrical connector system 20 of FIG. 2 substantially comprises the first electrical connector 28 and the second electrical connector 36. The first electrical connector 28 includes a group (e.g., a series, a set, a related plurality, or the like) of electrical connector pins 38, a first electrical housing 40, and an electrically conductive path 42, which may include any one or more of wires, traces, or some other conduit arranged to pass electric power or electrical signals.

In this embodiment, the group of electrical connector pins 38 includes at least three electrical connector pins 38. The three electrical connector pins 38 of FIG. 2 are configured to pass through the contamination barrier 30. Accordingly, the electrical connector pins 38 may be sharpened, pointed, or otherwise configured to facilitate passage of the pins through a particular barrier. In alternative embodiments, the number of electrical connector pins 38 may be of any quantity.

The first electrical housing 40 contains at least one portion of the electrically conductive path 42 and the electrical connector pins 38. The first electrical housing 40 may be made of an insulating material in the form of an epoxy, plastic, polymer, or some combination of insulating housing or coating materials. In addition, the first electrical housing 40 contains an insulating material 44. The insulating material 44 may provide electrical insulation, mechanical integrity, or other advantages. In the embodiment of FIG. 2, the insulating material 44 is used to insulate and hold the electrically conductive path 42 and the electrical connector pins 38 in place. The insulating material 44 may be an insulating material in the form of an epoxy, a plastic, a dielectric insulator, a polymer, or some combination of these or other insulting materials. In some cases, the first electrical housing 40, the insulating material 44, or both the first electrical housing 40 and the insulating material 44 are arranged to perform a coding feature to compatibly facilitate a cooperative mechanical coupling with a corresponding second electrical connector 36. The coding feature may include one or more shapes, structures, or other features that facilitate a proper alignment and coupling of first and second electrical connectors.

The electrically conductive path 42 may include or otherwise be coupled to the electrical connector pins 38. The electrically conductive path 42 facilitates passage of the electromagnetic drive signal produced by the magnetic sensing device 26 to the trackable structure 24.

The second electrical connector 36 includes a group of electrical connector pin receivers 46, a second electrical housing 48, and an electrically conductive path 42. The second electrical connector 36 is configured to electrically, mechanically, or electromechanically receive the first electrical connector 28.

In the embodiment of FIG. 2, the group of electrical connector pin receivers 46 includes at least three electrical pin receivers 46. In other embodiments, the second electrical connector may include any number of one or more electrical connector pin receivers 46. In some embodiments, there is a one-to-one correspondence between the number of electrical connector pins 38 and electrical connector pin receivers 46.

The three electrical pin receivers 46 in the embodiment of FIG. 2 are configured to receive the electrical connector pins 38. In addition, the three electrical pin receivers 46 are configured, formed, or otherwise arranged to reduce physical interference by material brought into the first and second connectors 28, 36 when the electrical connector pins 38 pass through the contamination barrier 30. For example, the electrical connector pins 38 may be formed with points arranged to pierce a contamination barrier 30 without separably tearing pieces of the contamination barrier 30 from the contamination barrier 30.

In the embodiment of FIG. 2, the number of electrical connector pin receivers 46 is equal to the number of electrical connector pins 38. In alternative embodiments, the number of electrical connector pin receivers 46 may be increased or decreased to any quantity or number. Furthermore, in alternative embodiments, the number of electrical connector pins 38 and electrical connector pin receivers 46 may be of different quantities or numbers.

The second electrical housing 48 includes some or all of one or more electrically conductive paths 42 and the electrical connector pin receivers 46. The second electrical housing 46 may be made of an insulating material in the form of an epoxy, plastic, polymer, or some combination of insulating housing or coating materials. The second electrical housing 46 of FIG. 2 contains an insulating material 44 used to insulate and hold the electrically conductive path 42 and the electrical connector pin receivers 46 in place. The insulating material 44 may be an insulating material in the form of an epoxy, a plastic, a dielectric insulator, a polymer, or some other insulating material.

In some cases, one or more portions of the medical electrical connector system 20 are formed from a magnetic shielding material. For example, some portion of the first electrical housing 40, the second electrical housing 46, or another portion of the medical electrical connector system 20 may include nickel, aluminum, brass, copper, iron, molybdenum, steel, or some other material that provides magnetic shielding. The materials may be pure or formed as an alloy. The materials may be formed as solid sheet or another solid arrangement, a mesh, a cage, a screen, or the like.

The electrically conductive path 42 couples to the electrical connector pin receivers 46. The electrically conductive path 42 is arranged to pass one or more electromagnetic drive signals produced by the magnetic field sensing device 26 to the trackable structure 24. The magnetic field sensing device 26 may be coupled to a power source or the magnetic field sensing device 26 may receive power by some other means.

The first and second electrical housings 40, 48 are configured in the embodiment of FIG. 2 to electrically, mechanically, or electromechanically interlock when the first and second electrical housings 40, 48 are joined together. The first and second electrical housings 40, 48 may interlock through means of interference fitting or some other method. In some embodiments, the first and second electrical housings 40, 48 are permanently affixed to each other. In other embodiments, the first and second electrical housings 40, 48 are configured to come apart. The electrically conductive path 42 is in some cases configured to be cut, for example, by the medical practitioner.

FIG. 2 shows several cross-sectional views of electrical connector pin configurations 54, 56, 58. The pin arrangements may be in line, circular, diamond, or with some other symmetry. The pin arrangements may in some cases be asymmetric. In some cases, the pin arrangements perform a coding feature to compatibly facilitate certain trackable structures 24 with certain magnetic field sensing devices 26.

In the cross sectional views 54, 56, 58, outer electrical connector pins 50 are configured to pass current in a first direction such that current passed in a center electrical connector pin 52 travels in an opposite direction. The current that passes through the outer electrical connector pins 50 may be a fraction of the current that passes through the center electrical connector pin 52. In these cases, for example, the fraction of the current that passes through each of the outer electrical connector pins 50 may be based on (e.g., inversely proportional to) the number of outer electrical connector pins 50. The total current of the outer electrical connector pins 50 may therefore be similar in value to the current that passes through the center electrical connector pin 52. Accordingly, in some embodiments, the volume of conductive material used to form the outer electrical connector pins 50 and the volume of conductive material used to form the electrically conductive paths 42 coupled to the outer electrical connector pins 50 may be correspondingly different from the volume of conductive material used to form the center electrical connector pin 52 and the volume of conductive material used to form the electrically conductive path 42 coupled to the center electrical connector pin 52, respectively.

In the embodiments of FIG. 2, placing the outer electrical connector pins 50 around the center electrical connector pin 52 reduces the external magnetic field produced by the first and second electrical connectors 28, 36. The outer connector pins 50 have a current that is similar in value and passes through the electrically conductive path 42 in the opposite direction to the current in the center electrical connector pin 52. This difference in direction of currents creates similar magnetic fields with opposite magnetic field directions. This difference in magnetic field directions causes the magnetic field of the outer electrical connector pins 50 to desirably cancel out some portion or all of the magnetic field produced by the center electrical connector pin 52. Thus, by surrounding a center electrical connector pin with a group of one or more outer electrical connector pins 50, and by passing a total current through the group of outer electrical connector pins 50 that is opposite in direction and similar in value to the current passed in the center electrical connector pin 52, the undesirable effects of external magnetic fields produced by first and second electrical connectors and their associated conductors can be reduced. As a result, there will be less magnetic interference in a magnetic field sensing device's position reading of a trackable structure.

FIG. 2 shows a cross-sectional view 60 of how the electrical connector pin receivers 46 couple to the electrical connector pins 38 in one embodiment. The electrical pin receivers 46 are configured to avoid physical interference due to material brought into the medical electrical connector system 20 when the electrical connector pins 38 pass through the contamination barrier 30. To reduce physical interference, the electrical pin receivers 46 allow a distal end (e.g., tip, point, edge, and the like) of the electrical connector pins 38 to pass by. The electrical connector pin receivers 46 then couple to an electrical contact portion of the electrical connector pins 38 past the distal end. In alternative embodiments, the electrical pin receivers 46 may be configured to receive the distal end of electrical connector pins 38 or receive the electrical connector pins 38 by some other means.

FIG. 3 is a first electrical connector embodiment and a second electrical connector embodiment that includes an ECG connector 62, 64 embodiment of a medical electrical connector system 20A embodiment. FIG. 3 is similar but different from the embodiment of the medical electrical connector system 20 of FIG. 2. The medical electrical connector system 20A of FIG. 3 substantially comprises the first electrical connector 28 and the second electrical connector 36. The electrical housings of the first electrical connector 28 and the second electrical connector 36 are omitted to simplify the illustration, but both housings may be configured similarly to or different from the first electrical housing 40 and the second electrical housing 48 in FIG. 2.

The first electrical housing 40 (not shown) contains individual ones or portions of the electrically conductive path 42 and the electrical connector pins 38. The first electrical housing 40 may be made of an insulating material in the form of an epoxy, a plastic, a polymer, or some other combination of insulating housing or coating materials. In addition, the first electrical housing 40 may contain an insulating material 44 used to insulate and hold the electrically conductive path 42 and the electrical connector pins 38 in place. The insulating material 44 may be an insulating material in the form of an epoxy, a plastic, a dielectric insulator, a polymer, or some other combination of insulating materials.

The electrically conductive path 42 couples to the electrical connector pins 38. The electrically conductive path 42 passes the electromagnetic drive signal produced by the magnetic sensing device 26 to the trackable structure 24.

In the embodiment of FIG. 3, the group of electrical connector pins 38 includes at least four electrical connector pins 38 and at least one signal electrical connector pin 62. The four electrical connector pins 38 are configured to pass through the contamination barrier 30. In alternative embodiments, the number of electrical connector pins 38 and signal electrical connector pins 62 may be of any quantity or number.

The second electrical connector 36 includes a group of electrical pin receivers 46, a second electrical housing 48, and an electrically conductive path 42. The second electrical connector 36 is configured to receive the first electrical connector 28.

The group of electrical pin receivers 46 includes at least four electrical pin receivers 46 in the embodiment of FIG. 3. The four electrical pin receivers 46 are configured to receive the electrical connector pins 38. In addition, the four electrical pin receivers 46 are configured to reduce physical interference by material brought into the first and second connectors 28, 36 when the electrical connector pins 38 pass through the contamination barrier 30.

In this embodiment, the number of electrical pin receivers 46 is equal to the number of electrical connector pins 38. In alternative embodiments, the number of electrical pin receivers 46 may be increased to any quantity or number. Furthermore, in alternative embodiments, the number of electrical connector pins 38 and electrical pin receivers may be of different quantities or numbers.

The medical electrical connector system 20A of FIG. 3 shows a signal electrical connector pin 62 that is added to the group of electrical connector pins 38 and not formed in the medical electrical connector system 20 of FIG. 2. Additional signal electrical connector pins 62 may be formed in other embodiments. The signal electrical connector pin 62 passes a current that differs from the other electrical connector pins. For example, the signal electrical connector pin 62 may pass one or more electrical signals such as power to an electro-cardiogram (ECG) or some other electrical medical device.

Similar to the group of electrical connector pins 38, a signal electrical pin receiver 64 is added to the other electrical pin receivers. The signal electrical pin receiver 64 is configured to receive the signal electrical connector pin 62. Similar to the signal electrical connector pin 62, the signal electrical pin receiver 64 is arranged to pass a current having different properties (e.g., voltage, current, frequency, data, and the like) compared to the other electrical connector pin receivers 46. For example, the signal electrical connector pin 64 may pass one or more signals to an ECG device, to some other medical device, or to some other electrical device.

FIG. 3 shows several cross-sectional views of electrical connector pin configurations 66, 68, 70. In the cross-sectional views 66, 68, 70, outer electrical connector pins 50 are configured to pass current in a direction that is opposite to the direction current is passing in the center electrical connector pin 52. The current that passes through each of the outer electrical connector pins 50 may be a fraction of the current that passes through the center electrical connector pin 52. The fraction of the current that passes through each of the outer electrical connector pins 50 may be proportional to the number of outer electrical connector pins 50. The total current passed via the outer electrical connector pins 50 may thereby be similar in value to the current that passes through the center electrical connector pin 52.

In embodiments of pin arrangements shown in FIG. 3, the outer electrical connector pins and outer electrical connector receivers 50 are placed around the center electrical connector pin and receiver 52. This placement reduces the external magnetic field surrounding these electrical connections. That is, surrounding the center electrical connector pin 52 and receiver 52 with the outer electrical connector pins 50 and receivers 50 reduces the external magnetic field produced when a low frequency alternating current drive signal is passed through the medical electrical connector system 20A. Thus, the magnetic interference from this external magnetic field is reduced causing less magnetic interference when the magnetic field sensing device 26 is used to generate information representing the position and location of the trackable structure 24.

In this embodiment, the signal electrical connector pin 62 and the signal electrical pin receiver 64 have been positioned for illustrative purposes. In alternative embodiments, the signal electrical connector pin 62 and pin receiver 64 may be located in some other manner.

For example, the signal electrical connector pin 62 and pin receiver 64 may be located closer to the center electrical connector pin 52 and pin receiver 52 than the outer electrical connector pins 50 and pin receivers 50, located farther from the center electrical connector pin 52 and pin receiver 52 than the outer electrical connector pins 50 and pin receivers 50, located a similar distance from the center electrical connector pin 52 and pin receiver 52 as the outer electrical connector pins 50 and pin receivers 50, located in some other manner, or positioned in some other manner.

In this embodiment, the signal electrical connector pin 62 and the signal electrical pin receiver 64 have the same cross-sectional area as the outer electrical connector pins 50 and pin receivers 52 for illustrative purposes. In alternative embodiments, the cross-sectional shape of the signal electrical connector pin 62 and receiver pin 64 may be square, rectangular, circular, triangular, or some other shape. In addition, the cross-sectional area of the signal electrical connector pin 62 and pin receiver 64 may be the same size as the outer electrical connector pins 50 and pin receivers 50, the same size as the center electrical connector pin 52 and pin receiver 52, or some other size.

The signal electrical connector pin 62 and the signal electrical pin receiver 64 may pass a current in the same direction as the outer electrical connector pins 50 and pin receivers 50, pass a current in the same direction as the center electrical connector pin 52 and pin receiver 52, or pass a current in some other direction.

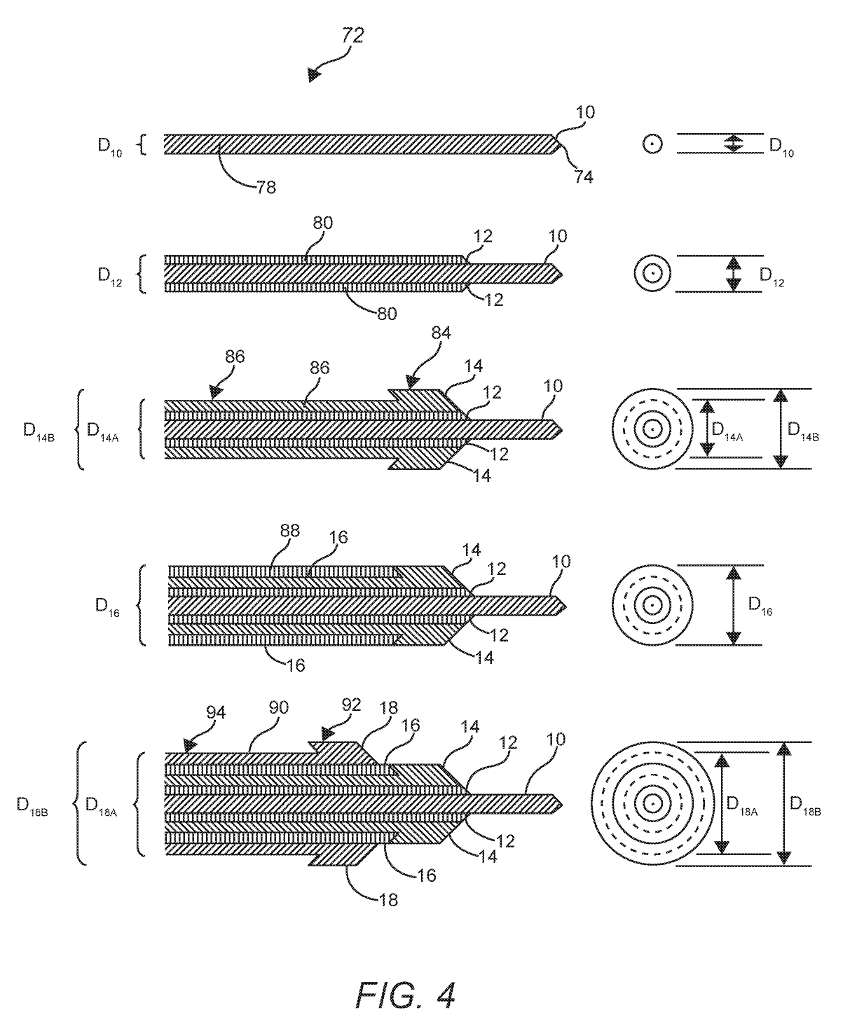

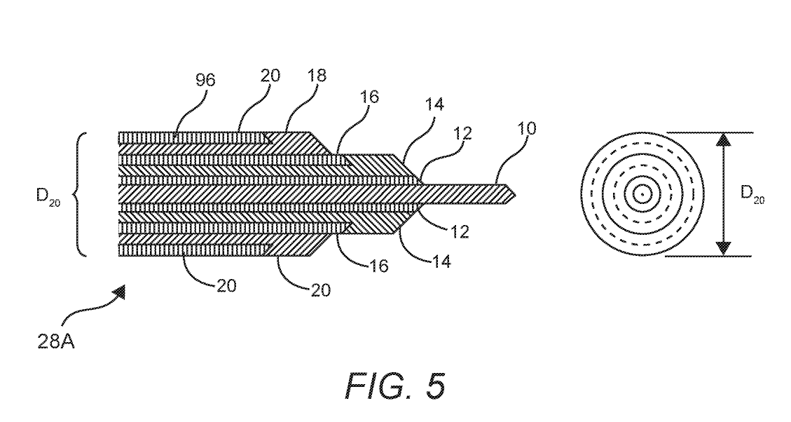

FIG. 4 is a method to produce one embodiment of a first electrical connector of a medical electrical connector system embodiment. FIG. 5 is a first electrical connector embodiment. Together, FIGS. 4 and 5 illustrate an alternative method embodiment to manufacture an embodiment of the first electrical connector 28A. This embodiment of the first electrical connector 28A includes an electrically conductive core 72, which may be rigid or flexible; first, second, and third coaxial insulator layers 80, 88, 96, which may be rigid or flexible; and first and second coaxial electrically conductive shield layers 82, 90, which may be rigid or flexible. The first and second coaxial electrically conductive shield layers 82, 90 include respective electrical contact areas 84, 92 and respective bodies 86, 94. The first electrical connector 28A is configured to pass through the contamination barrier 30.

In the method of FIGS. 4 and 5, an electrically conductive core 72 is formed or provided. The electrically conductive core 72 may be formed by an extrusion process or by another formation process of manufacturing. The electrically conductive core 72 includes a distal end 74, and a body 78. The electrically conductive core 72 may be made of copper, a copper-alloy, or another conductive material.

The body 78 is covered, coated, or otherwise formed to include a first coaxial insulator layer 80. The first coaxial insulator layer 80 may be altered, stripped, or otherwise formed to expose the distal end 74. The first coaxial insulator layer 80 fully or partially encompasses the body 76. The first coaxial insulator layer 80 may be made of an epoxy, a resin, a plastic, a rubber, or some other insulating material.

The first insulator layer 80 is covered, coated, or otherwise formed to include a first coaxial electrically conductive shield layer 82. The first coaxial electrically conductive shield layer 82 includes a first electrical contract area 84 and a first body 86. The first coaxial electrically conductive shield layer 82 may be made of copper, a copper-alloy, or another conductive material.

The first coaxial electrically conductive shield layer 82 is covered, coated, or otherwise formed to include a second coaxial insulator layer 88. The second insulator layer 86 may be altered, stripped, or otherwise formed to expose the first electrical contact area 84. The first electrical contact area 84 may have the same or different dimensions (e.g., diameter, thickness, or the like) as the first coaxial electrically conductive shield layer 82. The second coaxial insulator layer 88 fully or partially encompasses the first body 86. The second coaxial insulator layer 88 may be made of an epoxy, a resin, a plastic, a rubber, or some other insulating material.

The second coaxial insulator layer 88 is covered, coated, or otherwise formed to include a second coaxial electrically conductive shield layer 90. The second coaxial electrically conductive shield layer 90 includes a second electrical contact area 92 and a second body 94. The second electrical contact area 92 may have the same or different dimensions (e.g., diameter, thickness, or the like) as the second coaxial electrically conductive shield layer 90. The second coaxial electrically conductive shield layer 90 may be made of copper, a copper-alloy, or another conductive material.

The second coaxial electrically conductive shield layer 90 is covered, coated, or otherwise formed to include a third coaxial insulator layer 96. The third coaxial insulator layer 96 may be altered, stripped, or otherwise formed to expose the second electrical contact area 92. The third coaxial insulator layer 96 fully or partially encompasses the second body 94. The third coaxial insulator layer 96 may be made of an epoxy, a resin, a plastic, a rubber, or some other insulating material.

FIG. 4 shows cross-sectional views of this embodiment of the first electrical connector 28A at various steps of forming the first electrical connector 28A. FIG. 5 shows a cross-sectional view of the first electrical connector 28A after it is formed.

As shown in FIG. 5, the first electrical connector 28A has an overall diameter D20. The overall diameter D20 is in the range of 0.30 millimeters (mm)mm to 5 mm.

As shown in FIG. 4, the electrically conductive core 72 has a diameter D10 in the range of 0.1 mm to 1.5 mm. The first insulator layer 80 has a diameter D12 in the range of 0.11 mm to 2 mm. The first electrically conductive shield layer 82 has two diameters, the diameters of the body D14A and the first electrical contact area D14B. The diameter of the body D14A is in the range of 0.16 mm to 2.5 mm, and the diameter of the first electrical contact area D14B is in the range of 0.2 mm to 3.0 mm. The second insulator layer 88 has a diameter D16 in the range of 0.21 mm to 3.5 mm. The second electrically conductive shield layer 90 has two diameters, the diameters of the body D18A and the second electrical contact area D18B. The diameter of the body D18A is in the range of 0.26 mm to 4.0 mm, and the diameter of the second electrical contact area D18B is in the range of 0.3 mm to 4.5 mm. The third insulator layer 80 has a diameter D20 in the range of 0.31 mm to 5 mm. The thickness and diameters of the insulator layers 80, 88, 96 and the electrically conductive shield layers 82, 90 depend on the amount of current that will be passed through the first electrical connector 28A.

In alternative embodiments, the range of the diameters for the first electrical connector D20, the insulator layers D12, D16, D20, and the electrically conductive shield layers D14, D18 may be different in dimension. Likewise, in alternative embodiments, the overall diameter of the first electrical connector 28A may be different in dimension. The thickness and diameters of the insulator layers D12, D16, D20 and the electrically conductive shield layers D14, D18 depend on the amount of current that will be passed through the first electrical connector 28A.

In these embodiments, the smaller first electrical connector 28A embodiment allows for the first electrical connector 28A to pass through the contamination barrier with ease. The larger first electrical connector 28A embodiment allows for the first electrical connector 28A to be sturdier and less likely to break due to mechanical stresses, electrical stresses, electromechanical stresses, or some other stress. In addition, the larger first electrical connector 28A embodiment may pass a current larger than the smaller first electrical connector 28A embodiment. Thus, the smaller and the larger first electrical connector 28A embodiments may be utilized to deal with different contamination barriers, to deal with different stresses, to deal with different currents, or to deal with some other factor.

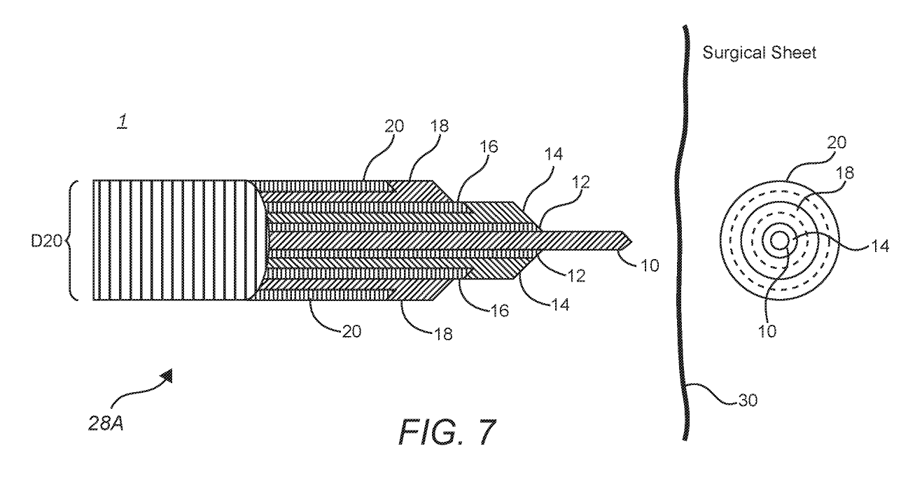

FIGS. 5 and 7 illustrate embodiments of the first electrical connector 28A produced by the method embodiment shown in FIG. 4. The electrical connector 28A produced by the method in FIG. 4 includes a flexible electrically conductive core 72, first, second, and third coaxial flexible insulator layers 80, 88, 96, and first and second coaxial flexible electrically conductive shield layers 82, 90. The first and second coaxial flexible electrically conductive shield layers 82, 90 include contact areas 84, 92 and bodies 86, 94. The first electrical connector 28A is configured to pass through the contamination barrier 30.

This embodiment of the first electrical connector 28 passes through the contamination barrier 30 using its distal end 74. The distal end 74 is configured to pass through the contamination barrier 30 by means of tearing, piercing, breaking, or some other way of passing through a physical barrier. This allows some or all of the first electrical connector 28A to pass through the contamination barrier 30 from the first space 32 to the second space 34 (FIG. 1).

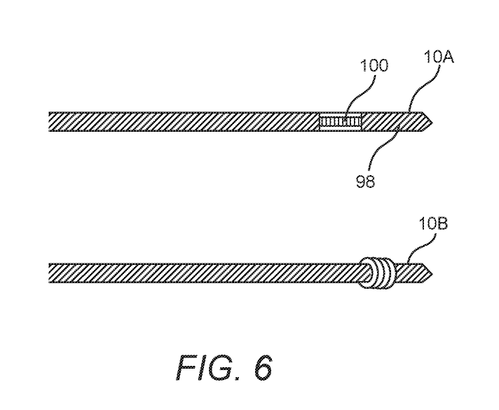

FIG. 6 shows two embodiments of the trackable structure 24. The trackable structure 24 may operate as an electromagnet that includes a trackable object 98 and a trackable conductor 100. The trackable object 98 may be all or a portion of a stylet, a catheter such as a Peripherally Inserted Central Catheter (PICC), a medical tube, a tracheal tube, a needle, a cannula, or some other structure. In some cases, the trackable object 98 is a hollow, tube-like device. In some cases, the trackable object 98 is an elongated, solid member. In some cases, the trackable object 98 takes another form. When so formed, the trackable structure may be configured as a medical device that will pass into the body of a patient during performance of a medical procedure.

The trackable conductor 100 receives an electromagnetic drive signal via the electrically conductive path 42, which cooperates with the trackable object 98 to produce an electromagnetic field detectable by the magnetic field sensing device 26. The magnetic field sensing device 26 senses the electromagnetic field produced by the trackable structure 24 and generates information representing the position and location of the trackable object 98. The trackable conductor 100 may be attached to or placed on the trackable object 98 by a channel, an opening, a space, a portion, or some other attachment or placement technique.

FIG. 7 is an embodiment of the first electrical connector 28A produced by the method embodiment shown in FIG. 4. The first electrical connector embodiment 28A is illustrated in side and front views. The first electrical connector 28A is arranged to pass through a contamination barrier such as the surgical sheet identified in FIG. 7.

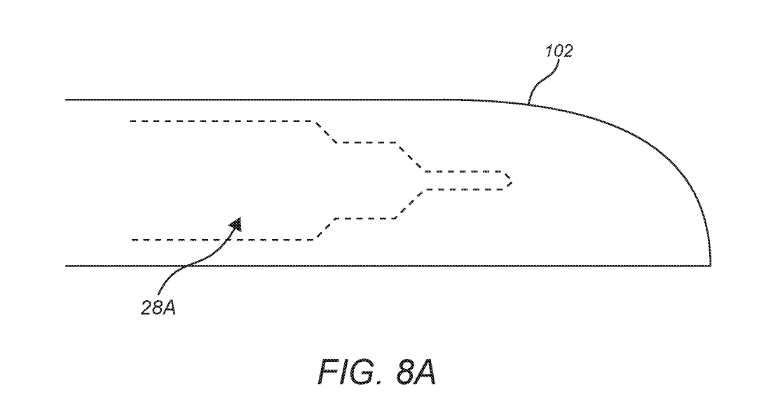

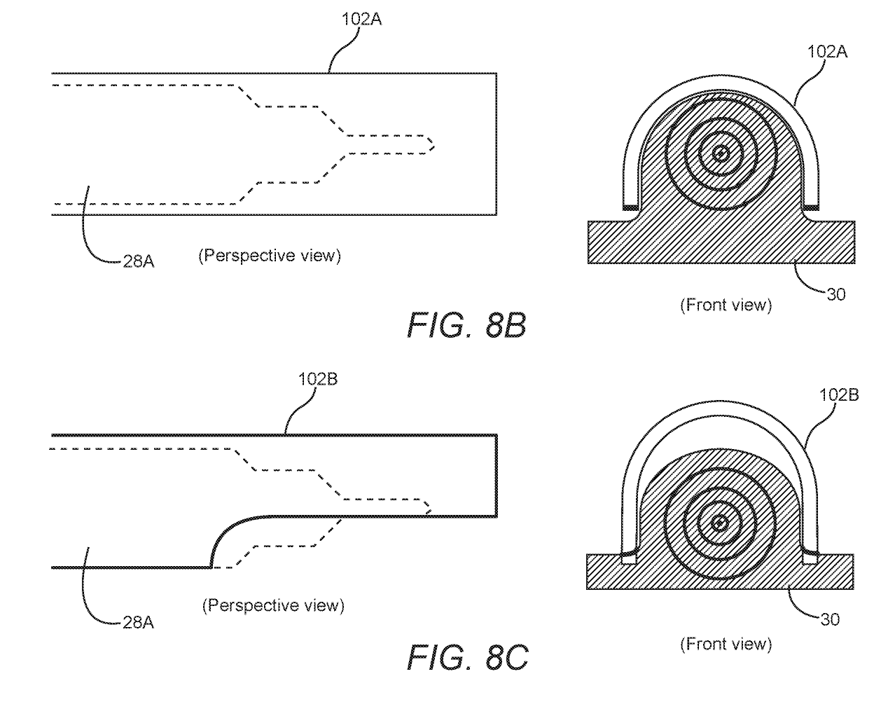

FIG. 8A is a shroud 102 embodiment for the first electrical connector embodiment 28A of FIGS. 5 and 7. The shroud 102 covers the first electrical connector 28A to protect the first electrical connector 28A from the outside world and external stresses. The external stresses may be electrical, mechanical, magnetic, chemical, or some other form of an external stress. The shroud 102 may also be arranged for other reasons such as to protect a medical practitioner from injury caused, for example, by a sharpened portion of the first electrical connector 28A. The shroud 102 may be made of a polymer, plastic, or some other material used to make a protective covering. In addition, this embodiment of the shroud 102 protects the first electrical connector 28A from contaminates before it passes through the contamination barrier 30. Thus, utilizing a shroud 102 to protect a first electrical connector 28A reduces the chance of contamination from reaching a second space 34 associated with a contamination barrier 30.

FIGS. 8B and 8C are additional shroud embodiments 102A, 102B along the lines of the embodiment in FIG. 8. The materials used to form the shrouds 102A, 102B, and the purposes for including the shrouds 102A, 102B, may be the same or similar to those materials and purposes associated with the shroud 102 of FIG. 8. A perspective view and a front view of each of shrouds 102A, 102B is shown in FIGS. 8B, 8C respectively.

The shroud embodiment 102A of FIG. 8B has a truncated leading edge, which in exemplary cases may be formed as a half cylinder cut along a horizontal plane. The shroud embodiment 102B of FIG. 8C has a truncated leading edge, which in exemplary cases may be formed as a half cylinder cut along a horizontal plane with a further cutaway portion on the leading edge. Other embodiments are also contemplated. In at least some embodiments, it is desirable when the shroud 102, 102A, 102B portion of the first connector assembly is arranged to naturally slide easily against the contamination barrier 30. For example, the drape may be held against and electrical receptacle such as the second electrical connector 36. In this way, the distal end 74 of the first electrical connector 28A penetrates the contamination barrier 30. In some cases, the distal end 74 of the first electrical connector 28A is suitably sharpened to pierce the contamination barrier 30 rather than stretching it. Along these lines, the electrical receptacle (e.g., a second electrical connector 36) maybe formed with significant friction between the contamination barrier 30 and the receptacle. An arrangement that includes significant friction between the contamination barrier 30 and the receptacle, but not between the contamination barrier 30 and the shroud 102, 102A, 102B reduces the likelihood of the contamination barrier 30 collecting, gathering, or otherwise “bunching up” in front of the shroud 102, 102A, 102B.

The shrouds 102, 102A, 102B of FIGS. 8A-8C are optional. In some cases, the first shroud embodiments 102, 102A, 102B may be arranged to perform a coding feature to compatibly facilitate a cooperative coupling with a suitable receptacle such as a second electrical connector 36. The coding feature may include any number of shapes, structures, or other visual or mechanical features that facilitate a proper alignment and coupling of first and second electrical connectors.

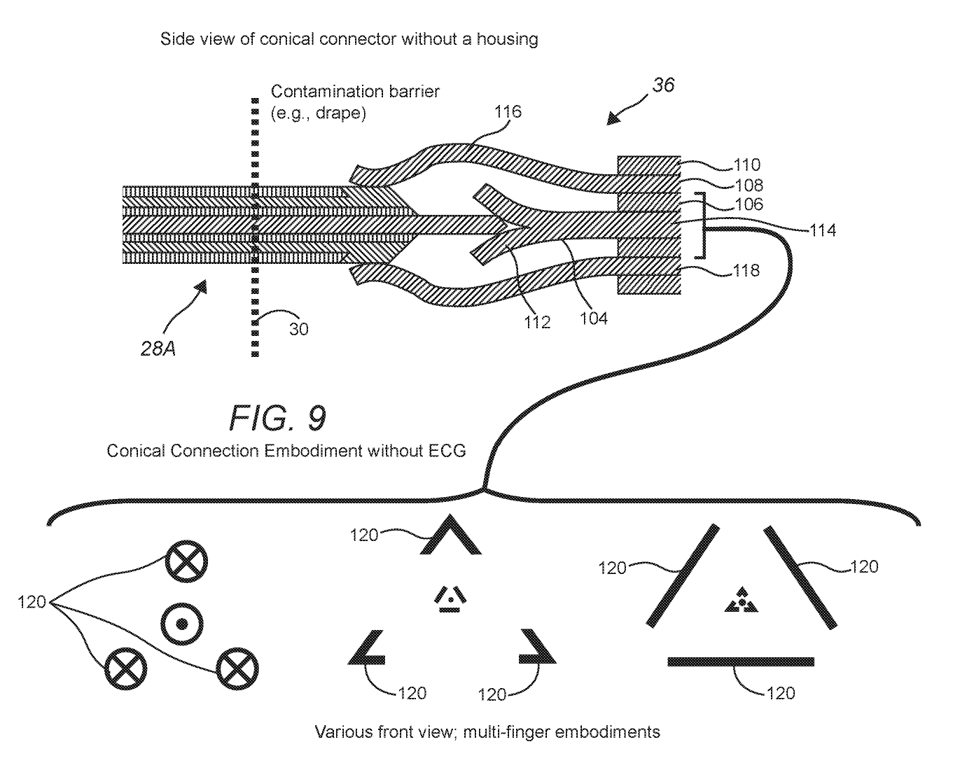

FIG. 9 is one embodiment of the second electrical connector 36. The second electrical connector 36 is configured to receive the first electrical connector 28A. The second electrical connector 36 includes a first electrically conductive multi-leaf receiver 104, a first flexible insulator layer 106, a second electrically conductive receiver 108, and a second flexible insulator layer 110.

The first electrically conductive multi-leaf receiver 104 includes a first electrical receiver end 112 and a first body 114. The first electrical receiver end 112 includes at least three electrically conductive leafs 112. The three electrically conductive leafs 112 are configured to receive the distal end 74 of the electrically conductive core 72. The first flexible insulator layer 106 is attached to and encompasses the first body 114. The second electrically conductive receiver 108 includes a second electrical receiver end 116 and a second body 118. The second electrical receiver end 116 is configured to receive and electrically contact the first electrical contact area 84. The second flexible insulator layer 110 is attached to and encompasses the second body 118. The first and second bodies may be made of a flexible conductive material. In an alternative embodiment, the multi-leaf receiver 104 may include more or less than three electrically conductive leafs, one solid receiver such that it is an infinite number of leafs, or some other suitable structure arranged to make electrical contact with the electrically conductive core 72.

This embodiment of the second electrical connector 36 in FIG. 9 may be manufactured using a corresponding method embodiment as the first electrical connector 28A in FIG. 4, or the electrical connector 36 may be made using a different method. That is, the insulator layers 106, 110, the electrical receiver ends 112, 116, and the first and second bodies 114, 118 may be formed as cooperating layers until this embodiment of the second electrical connector 36 is produced.

For example, in one non-limiting and exemplary method, a first electrically conductive multi-leaf receiver 104 is formed. The first electrically conductive multi-leaf receiver 104 may be formed by an extrusion process or by another formation process of manufacturing. The first electrically conductive multi-leaf receiver 104 has a first body 114 and a first electrical receiver end 112.

The first body 114 is then covered in a first flexible insulator layer 106. The first flexible insulator layer 106 encompasses the first body 114. The first flexible insulator layer 106 may be made of an epoxy, a resin, a plastic, a rubber, or some other insulating material.

The first flexible insulator layer 106 is then covered by a second electrically conductive receiver 108, the second electrically conductive receiver 108 having a second body 118 and a second electrical receiver end 116. The second electrically conductive receiver 108 may be made of a copper, a copper-alloy, or some other conductive material. The second body 118 encompasses the first flexible insulator layer 106.

The second body 118 is then covered in a second coaxial flexible insulator layer 110. The second coaxial flexible insulator layer 110 encompasses the first body 118. The second coaxial flexible insulator layer 110 may be made of an epoxy, a resin, a plastic, a rubber, or some other insulating material.

In FIG. 9, several different multi-finger front view embodiments are illustrated as exemplary arrangements of the electrical receiver ends 112, 112. In the front view embodiments, various ones of the conductive fingers 120 are shown in different configurations, wherein the fingers are arranged to cooperate in the formation of suitable electromechanical unions with structures of the first electrical connector 28A. The embodiments are non-limiting, and three examples are illustrated for simplicity in the description. Many other arrangements are also contemplated.

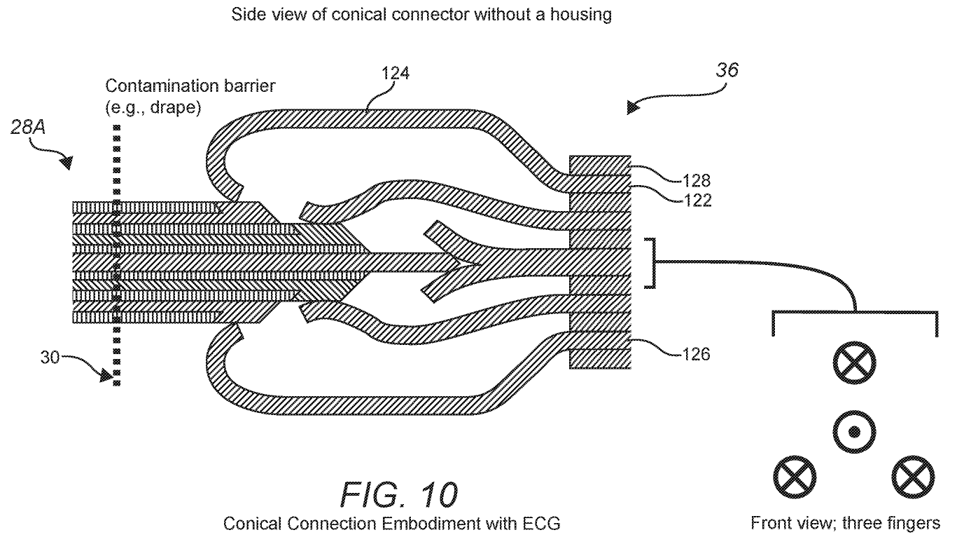

The various configurations of FIG. 9, and of FIG. 10 described herein, represent particular symmetry that may be suitable for coaxial, triaxial, or the like approaches. These embodiments are different from the more discrete approaches suggested in FIGS. 2 and 3, which may be particularly suited for twisted wire cabling.

FIG. 10 is a multipart connector to electrically couple a magnetic field sensing device 26 to a trackable structure 24. In the embodiment of FIG. 10, the second electrical connector 36 is configured to receive the first electrical connector 28A. The first electrical connector 28A passes through the contamination barrier 30. The second electrical connector 36 includes a first electrically conductive multi-leaf receiver 104, a first flexible insulator layer 106, a second electrically conductive receiver 108, a second flexible insulator layer 110, a third electrically conductive receiver 122, and a third flexible insulator layer 128.

The first electrically conductive multi-leaf receiver 104 includes a first electrical receiver end 112 and a first body 114. The first electrical receiver end 112 includes at least three electrically conductive leafs 120. The three electrically conductive leafs 112 are configured to receive the distal end 74 of the electrically conductive core 72. The first flexible insulator layer 106 is attached to and encompasses the first body 114. The second electrically conductive receiver 108 includes a second electrical receiver end 116 and a second body 118. The second electrical receiver end 116 is configured to receive and electrically contact the first electrical contact area 84. The second flexible insulator layer 110 is attached to and encompasses the second body 118. The third electrically conductive receiver 122 includes a third electrical receiver end 124 and a third body 126. The third electrical receiver end 124 is configured to receive the second electrical contact area 92. The third flexible insulator layer 128 is attached to and encompasses the third body 126.

This embodiment of the second electrical connector 36 in FIG. 10 is manufactured using a similar method embodiment as the first electrical connector 28A in FIG. 4 and results in a similar embodiment of the first electrical connector 28A in FIG. 9. That is, the insulator layers 106, 110, 128, the electrical receiver ends 112, 116, 124, and the first, second, and third body 114, 118, 126 are cooperatively layered until this embodiment of the second electrical connector 36 is formed.

More specifically, in this method, a first electrically conductive multi-leaf receiver 104 is formed. The first electrically conductive multi-leaf receiver 104 may be formed by an extrusion process or by another formation process of manufacturing. The first electrically conductive multi-leaf receiver 104 has a first body 114 and a first electrical receiver end 112.

The first body 114 is then covered in a first flexible insulator layer 106. The first flexible insulator layer 106 encompasses the first body 114. The first flexible insulator layer 106 may be made of an epoxy, a resin, a plastic, a rubber, or some other insulating material.

The first flexible insulator layer 106 is then covered by a second electrically conductive receiver 108, the second electrically conductive receiver having a second body 118 and a second electrical receiver end 116. The second electrically conductive receiver 108 may be made of a copper, a copper-alloy, or some other conductive material. The second body 118 encompasses the first flexible insulator layer 106.

The second body 118 is then covered in a second coaxial flexible insulator layer 110. The second coaxial flexible insulator layer 110 encompasses the first body 118. The second coaxial flexible insulator layer 110 may be made of an epoxy, a resin, a plastic, a rubber, or some other insulating material.

The second coaxial flexible insulator layer 110 is then covered by a third electrically conductive receiver 122. The third electrically conductive receiver 122 includes an electrical receiver end 124 and a third body 126. The third electrically conductive receiver 122 may be made of copper, a copper-alloy, or some other conductive material.

The third body 126 is then covered by a third coaxial flexible insulator layer 128. The third coaxial flexible insulator layer 128 encompasses the third body 126. The third coaxial flexible insulator layer 128 may be made of an epoxy, a resin, a plastic, a rubber, or some other insulating material.

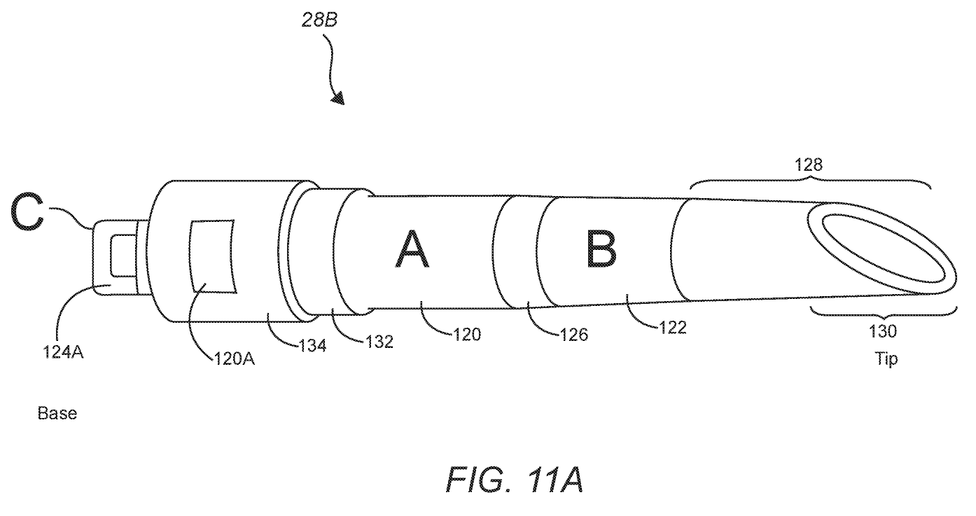

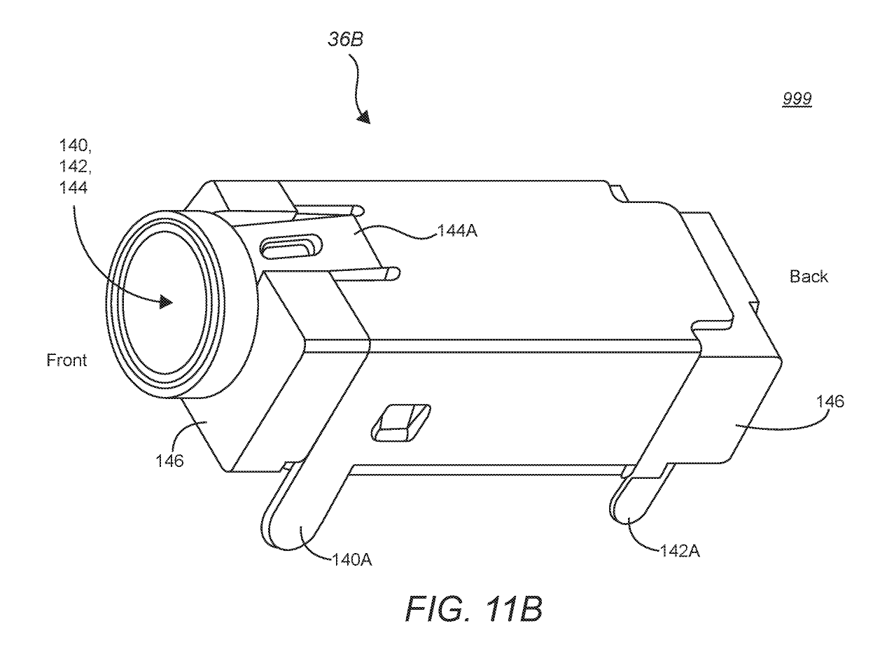

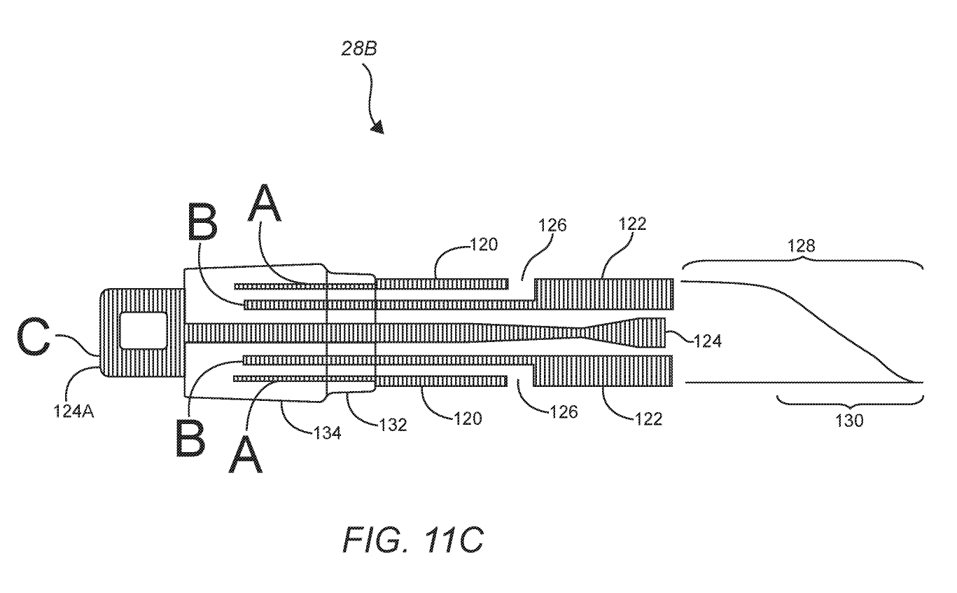

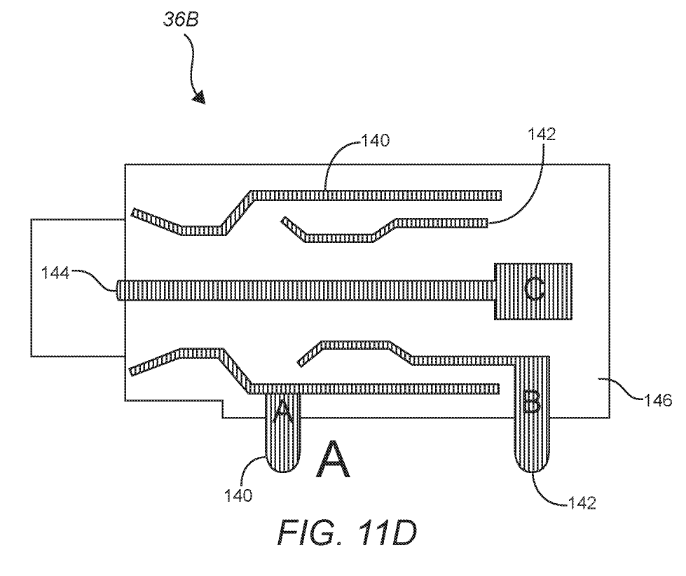

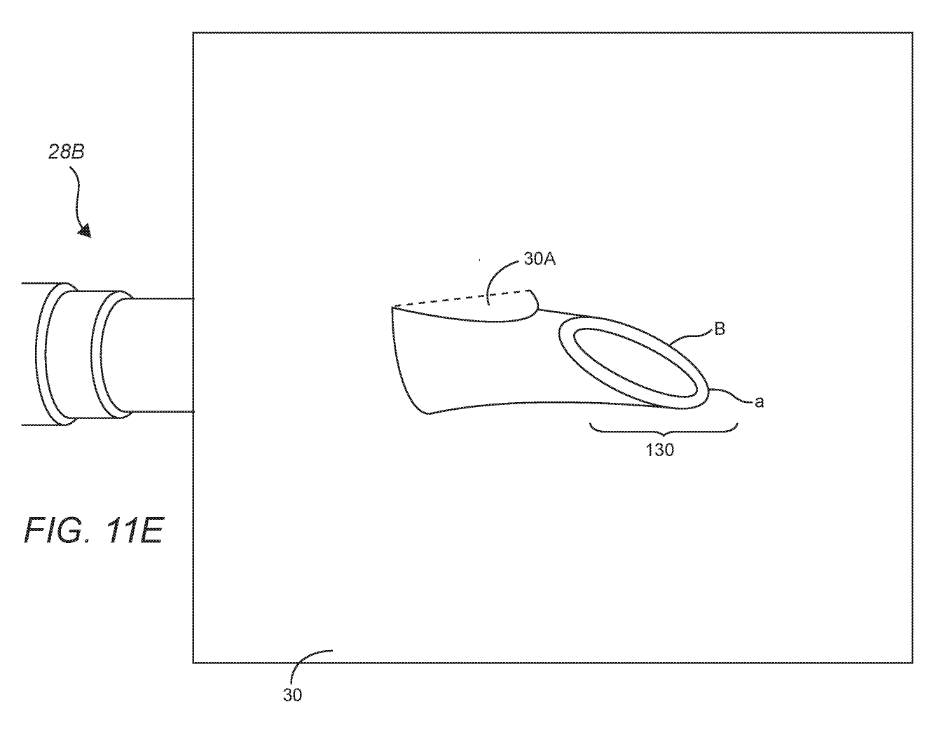

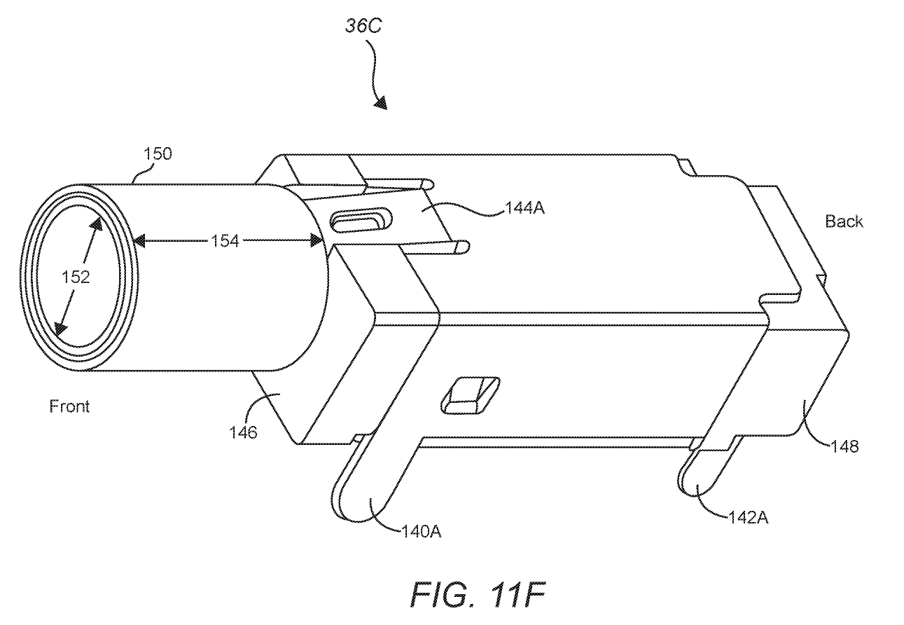

FIGS. 11A-11B are first and second electrical connector embodiments 28B, 36B, respectively, that cooperate in a connector system such as the medical electrical connector system 20 embodiment of FIG. 2.

The first electrical connector 28B of FIG. 11A may be referenced with a proximal end, or “base” and a distal end, of “tip,” which are labeled in FIG. 11A and used in the description herein. Generally speaking, a practitioner deploys the connector system by grasping the base of the first electrical connector 28B and advancing the tip of the first electrical connector 28B through the body of the second electrical connector 36B thereby forming a plurality of separate and distinct electrical connections.

The first electrical connector 28B of FIG. 11A has a substantially cylindrical barrel in which a cross-section of the barrel's linear dimension is substantially circular. In other embodiments, the cross-section of the linear dimension may be square, rectangular, hexagonal, octagonal, or some other shape. The cross-sectional shape of the first electrical connector 28B of FIG. 11A and the cross-sectional shape of the second electrical connector 36B of FIG. 11B are arranged to mate in mechanical and electrical cooperation.

The substantially cylindrical barrel of the first electrical connector 28B embodiment of FIG. 11A includes a first electrical contact surface 120 and a second electrical contact surface 122 separated by an insulator/housing 126. The first and second electrical contact surfaces are labeled “A” and “B,” respectively, in FIG. 11A to aid in understanding FIGS. 11A-11E. A leading insulator/housing 128 is formed as a leading structure at the tip of the first electrical connector 28B. The leading insulator/housing 128 includes an integrated piercing structure 130. The substantially cylindrical barrel portion of first electrical connector 28B of FIG. 11A may include an optional securing mechanism 132 coupling structures of the barrel to each other, to an insulator/housing base 134, or in another arrangement. The securing mechanism, when included in some embodiments, may be a threaded ring, a compression or friction fitting, a wedge or shim, an adhesive structure, or some other securing means. To this end, various other means of providing structural integrity to the first electrical connector 28B, which do not depart from the inventive aspects of the connector, are also contemplated.

Optionally, certain base contacts may be arranged in or in association with the insulator/housing base 132. The base contacts may include surfaces, loops, pigtails, posts, tabs, nodes, or other structures to which an electrical conduit such as a wire may be attached such as by soldering, crimping, or the like. In this way, electrical signals may be independently pass, uni-directionally or bi-directionally, from one electronic device (e.g., a magnetic field sensing device 26 as in FIG. 1), through the first electrical connector 28B, through the cooperating second electrical connector 36B, and to another electronic device (e.g., a trackable structure 24 as in FIG. 1).

The first electrical connector 28B of FIG. 11A includes three separate and distinct electrical signal paths. First electrical signals may pass from a first electronic device, through a first base contact 120A, through electrical contact A 120, through a first electrical contact A 140 in the second electrical connector 36B, through a first base contact 140A in the second electrical connector 36B, and to the other electronic device. Correspondingly, second electrical signals may pass from the first electronic device, through a second base contact 122A (not shown), through electrical contact B 122, through a second electrical contact B 142 in the second electrical connector 36B, through a second base contact 142A in the second electrical connector 36B, and to the other electronic device. And further still, third electrical signals may pass from the first electronic device, through a third base contact 124A, through an electrical contact C 124 (FIG. 11C), through a third electrical contact C 144 in the second electrical connector 36B, through a third base contact 144A in the second electrical connector 36B, and to the other electronic device.

The second electrical connector 36B of FIG. 11B is arranged to electrically and mechanically receive the first electrical connector 28B of FIG. 11A. Operatively, the leading insulator/housing 128 at the tip of the first electrical connector 28B is received at the front of the second electrical connector 36B. The tip of the first electrical connector 28B is then advanced within and toward the back of the second electrical connector 36B. When fully inserted, the insulator/housing base 134 of the first electrical connector 28B abuts the front of the second electrical connector 36B. In addition, when fully inserted, the electrical contact A 120 of the first electrical connector 28B is in electrical contact with an electrical contact A 140 (FIG. 11D) of the second electrical connector 36B; the electrical contact B 122 of the first electrical connector 28B is in electrical contact with an electrical contact B 142 (FIG. 11D) of the second electrical connector 36B; and the electrical contact C 124 of the first electrical connector 28B is in electrical contact with an electrical contact C 144 of the second electrical connector 36B. Electrical contacts 140, 142, 144 are accessible internal to the second electrical connector 36B via an aperture at the front of the second electrical connector 36B

FIGS. 11C-11D are cross-sections of the first and second electrical connector embodiments 28B, 36B, of FIGS. 11A-11B, respectively. The cross-section is taken across a linear dimension of the electrical connectors.

In some embodiments, one or more of the electrical contacts A, B, C, 120, 122, 124 are formed having a generally cylindrical shape. In some embodiments, one or more of the electrical contacts A, B, C, 120, 122, 124 are formed of multiple portions. The electrical contacts may be defined as having particular linear dimensions, curvilinear dimensions, or some other shape and dimension. In some embodiments, the exposed portion of electrical contact A 120 has substantially same exposed portion as the exposed portion of electrical contact B 122. In other cases, either electrical contact A 120 or electrical contact B 122 is formed having a greater exposed portion. In some cases, the size of the contact and in the alternative or in addition the size of the exposed portion of the contact is desirably controlled to limit the amount of stray electromagnetic energy that escapes the electrical connector system.

In the embodiment of FIGS. 11C-11D, when the first electrical connector 28B is coupled to the second electrical connector 36B, the electrical contact A 120 is received by the electrical contact A 140. The receiving electrical contact A 140 may be flexibly arranged to facilitate both mechanical and electrical coupling. Correspondingly, the electrical contact B 122 is received by the electrical contact B 142, and the receiving electrical contact B 142 may be flexibly arranged to facilitate both mechanical and electrical coupling. In the coupling as illustrated, the electrical contact C 124 of the first electrical connector 28B is arranged to mechanically and electrically receive the electrical contact C 144 of the second electrical connector 36B.

FIG. 11E illustrates the first connector embodiment 28B of FIG. 11A passing through a contamination barrier 30. As evident in FIG. 11E, when the piercing structure 130 passes through the contamination barrier 30, a portion (e.g., a flap 30A) of the contamination barrier 30 is cut and moved out of the way of the advancing first electrical connector 28B. The shape of the contamination barrier portion that is moved is generally based on the shape of the piercing structure 130, and due to the shape of the piercing structure 130, the risk of a small piece of the contamination barrier 30 separating from the larger contamination barrier 30 is reduced.

The piercing structure 130 may be arranged to pierce a contamination barrier 30 in several ways. As illustrated, for example, the piercing structure 130 in FIG. 11E is sharpened. A desirable sharpness may be incorporated or otherwise implemented in the piercing structure 130 in several ways. In some embodiments, the piercing structure 130 is formed with a first sharpened edge having angle α, which is an angle of the cut through the wall of the piercing structure 130. In these or in other embodiments, the piercing structure 130 is formed having a swept-back (e.g., tapered) cut of angle β, which is an angle of the cut through the entire diameter of the piercing structure 130. In still other embodiments, the piercing structure 130 is formed having with a desired radius of the tip of the piercing structure 130, a desired hardness of the material of the piercing structure 130, a selected number and pattern of serrations, and in other ways. In some embodiments along the lines of piercing structure 130 of FIG. 11E, a first angle α of the cut through the wall of the piercing structure 130 is between 20 and 50 degrees. In these or other embodiments, a second angle μ of the cut through the entire diameter of the piercing structure 130 is between 35 and 65 degrees. In some embodiments, the first angle α and the second angle β are substantially the same. In these or in other embodiments, the piercing structure 130 may be formed having a double edge.

FIG. 11F illustrates another embodiment of the second electrical connector 36C. The second electrical connector 36C of FIG. 11F is along the lines of the second electrical connector 36B of FIG. 11B, and like structures of the second electrical connector 36C are given the same reference numbers and not further described for simplicity.

The second electrical connector 36C of FIG. 11F includes an extended front receiver portion 150, which is arranged to receive the leading insulator/housing 128 of the first electrical connector 28B. The extended front receiver portion 150 has a diameter 152 that is shorter than its length 154. The extended front receiver portion 150 is formed in this manner to reduce the likelihood of a contamination barrier flap 30A contacting a third electrical contact C 144.

FIG. 11G illustrates a portion of a cooperative coupling method between a first electrical connector 28B of FIG. 11A and a second electrical connector 36G of FIG. 11F. In the figure, the leading insulator/housing 128 of the first electrical connector 28B is advancing through a contamination barrier 30. As the leading insulator/housing 128 moves forward, a contamination barrier flap 30A is formed and moved out from the path of the first electrical connector 28B. Due to the arrangement of the extended front receiver portion 150 of the second electrical connector 36C, the chance that the contamination barrier flap 30A will contact the third electrical contact C 144 is reduced, and correspondingly, the chance that the third electrical contact C 144 is bent, misaligned, or otherwise prevented from cooperatively contacting the third electrical receiver end 124 of the first electrical connector 28B are also reduced.

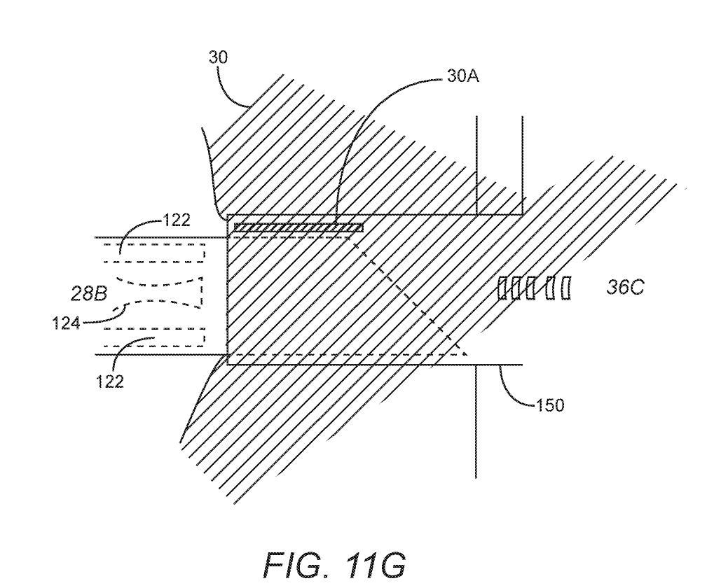

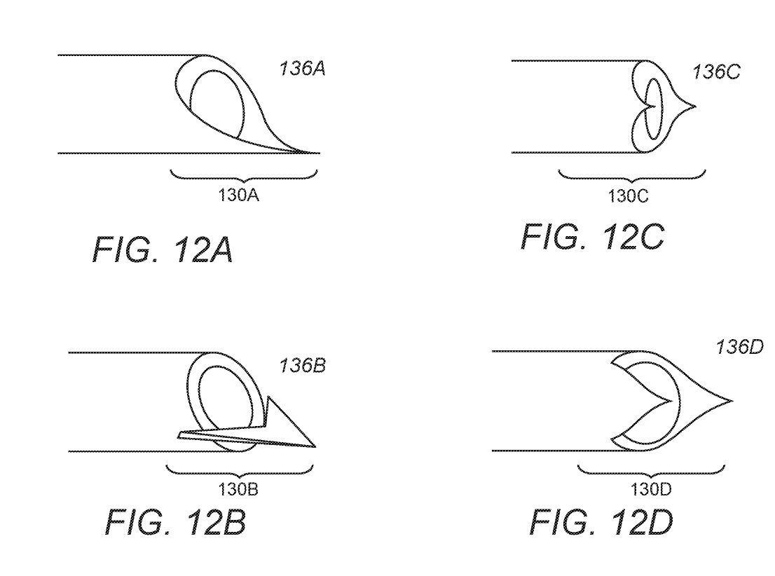

FIGS. 12A-12D are piercing structure embodiments 130A-130D. The embodiments of FIGS. 12A-12D are exemplary and non-limiting. In the embodiment of FIG. 12A, for example, a first electrical connector 28B (FIG. 11A) is formed having a leading insulator/housing 128 with a trocar-style piercing embodiment 130A. The first electrical connector 28B (FIG. 11A) is formed having a leading insulator/housing 128 with a bladed piercing structure embodiment 130B in FIG. 12B. The blade in FIG. 12B may be formed of stainless steel or another material, and the blade may be attached or otherwise integrated with the piercing structure in any known way. In FIG. 12C, the leading insulator/housing 128 has a rounded dual piercing structure embodiment 130C, and in FIG. 12D, the leading insulator/housing 128 has a tapered (e.g., beveled) dual piercing structure embodiment 130D.

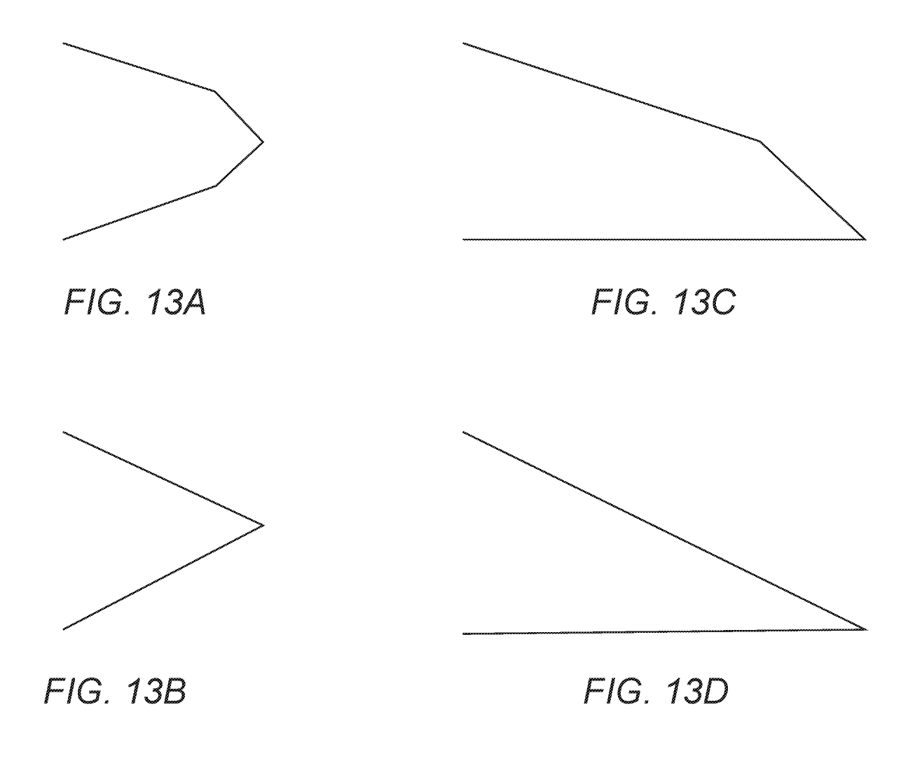

FIGS. 13A-13D are optional piercing structure sharpened edge embodiments. In order to improve the ease in which the first electrical connector 28B (FIG. 11A) pierces, cuts, or otherwise passes through the contamination barrier, a leading edge of the piercing structure may be optionally and desirably formed. The embodiments of FIGS. 13A-13D are non-limiting and exemplary.

In FIG. 13A, a leading edge of a piercing structure 130 (FIG. 11A) may formed having a double-beveled, dual edge 136A. In FIG. 13B, the leading edge of a piercing structure 130 (FIG. 11A) may be formed having single-beveled dual edge 136B, and in FIG. 13C, the leading edge may be formed having a double-beveled single edge 136C. FIG. 13D illustrates a leading edge of a piercing structure embodiment having a simple beveled edge 136D.

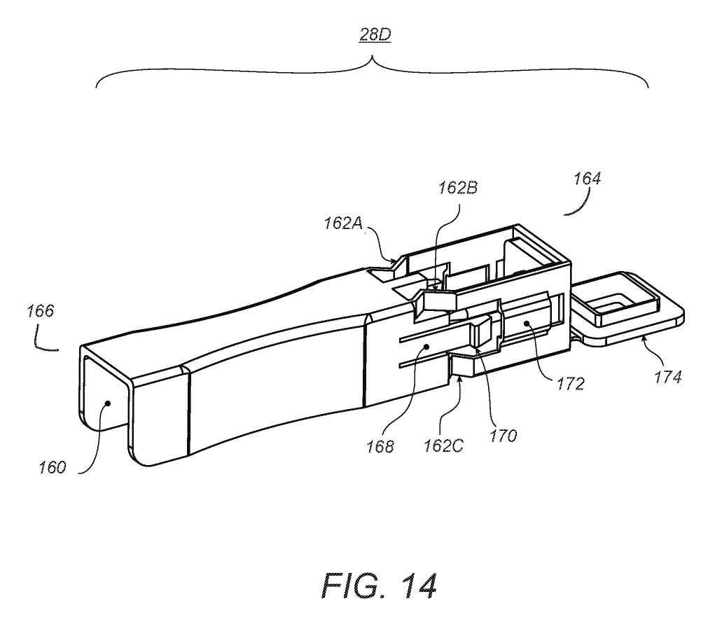

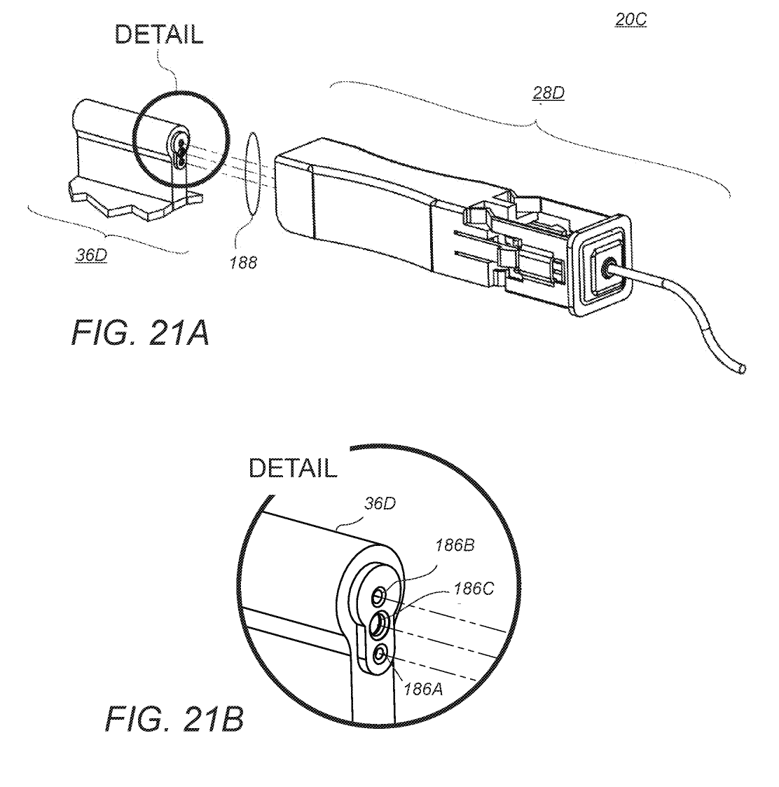

FIGS. 14-25C illustrate first and second electrical connector embodiments of a two-stage electrical connector system 20C, which is shown with particular detail in FIG. 21A.

FIG. 14 is a first electrical connector embodiment 28D. An entry side 160 is identified in the front portion 166 of the first electrical connector embodiment 28D. In use, the entry side 160 of the first electrical connector embodiment 28D would be placed in contact with a contamination barrier 30 (not shown in FIG. 14), and a piercing portion of the first electrical connector embodiment 28D would penetrate the contamination barrier 30.

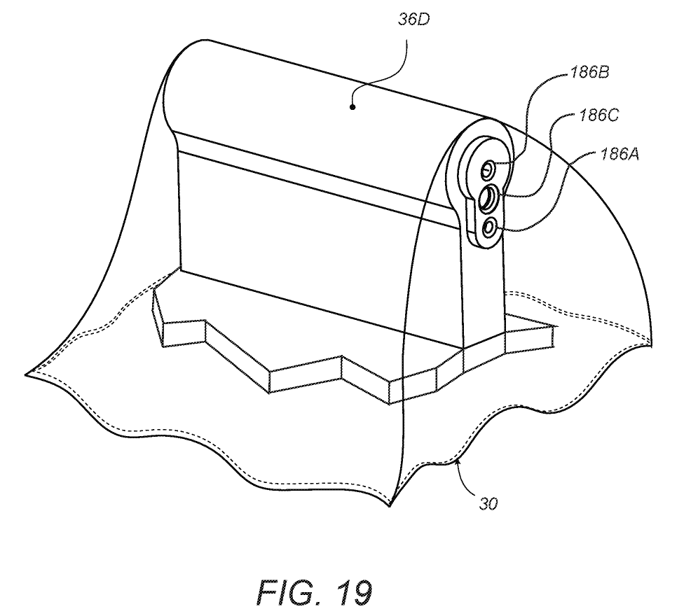

In some cases, live hinges 162A, 162B, 162C are formed in the first electrical connector embodiment 28D. The live hinges 162A, 162B, 162C function as springs that permit a rear portion 164 of the first electrical connector embodiment 28D housing to move relative to the front portion 166. This motion provides flexibility during construction of the first electrical connector embodiment 28D, during electromechanical coupling or decoupling of the first electrical connector embodiment 28D and a cooperating second electrical connector portion 36D (FIG. 19), strain relief for cabling (e.g., wire, wires, tethers, and the like), and other benefits.

The first electrical connector embodiment 28D includes one or more cantilever arms 168 that are arranged to align the rear portion 164 of the first electrical connector embodiment 28D to the front portion 166. One cantilever arm 168 is shown in FIG. 14 proximal to live hinge 162B, and in at least some cases, a second cantilever arm 168 (not shown) is arranged on the other side of the first electrical connector embodiment 28D proximal to live hinge 162A. In addition to provide a guidance function, one or more cantilever arms 168 are further arranged to establish or otherwise perform a positive mechanical locking function when the rear portion 164 of the first electrical connector embodiment 28D is advanced toward the front portion 166.

The cantilever arm 168 embodiment of FIG. 14, formed on the front portion 166 of the first electrical connector embodiment 28D, is arranged having a locking surface 170 formed thereon. In these cases, the rear portion 164 of the first electrical connector embodiment 28D is arranged with a locking receiver 172 feature that will cooperate with the locking surface 170. As described herein, when the rear portion 164 of the first electrical connector embodiment 28D is advanced toward the front portion 166, live hinges 162A, 162B, 162C flexibly “collapse” thereby allowing the locking surface 170 to positively engage the locking receiver 172. Other locking mechanisms and other types of hinge and non-hinge flexibility mechanisms are contemplated.

In at least some embodiments, an optional rear lid 174 is arranged for cooperation at the rear portion 164 of the first electrical connector embodiment 28D. The rear lid 174 may provide cabling strain relief, protection from the ingress of foreign material into the housing of the first electrical connector embodiment 28D, structural stability for the first electrical connector embodiment 28D, and other operations and benefits. The optional rear lid 174 may be flexibly attached to the rear portion 164 of the first electrical connector embodiment 28D, or the optional rear lid 174 may be a separate and distinct structure from the first electrical connector embodiment 28D. The optional rear lid 174 has any desirable shape and may incorporate additional features.

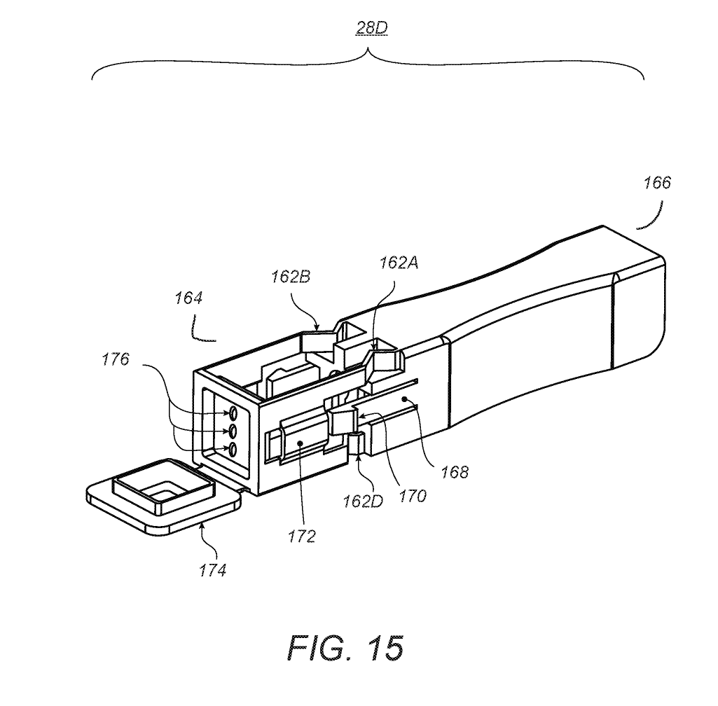

FIG. 15 is the first electrical connector embodiment 28D of FIG. 14 viewed from a second perspective. In the second perspective, the rear portion 164 and the front portion 166 of the first electrical connector embodiment 28D are evident as viewed from the opposite side as in FIG. 14. In this figure, live hinges 162A and 162B are shown, and a fourth live hinge 162D is presented. In other embodiments of electrical connectors along the lines of the first electrical connector embodiment 28D, a different number of hinges, a different configuration of hinges, and different structure and structural operation of flexible members may be formed. Also evident in FIG. 15 are a second cantilever arm 168, a second locking surface 170 and a second locking receiver 172, the operation of which has been described.

Viewed from the second perspective, in proximity to the optional rear lid 174, one or more electrical contact apertures 176 are formed. The number, shape, and other features of the apertures may be different in other embodiments. For example, rather than round holes, the apertures may be square, hexagonal, or with some other shape. The arrangement of a plurality of apertures may additionally or alternatively be different in other embodiments. For example, the apertures may be sized differently as a keying mechanism, the apertures may be arranged at different distances or in a different pattern as a keying mechanism, still other embodiments may arrange the aperture in any desirable configuration.

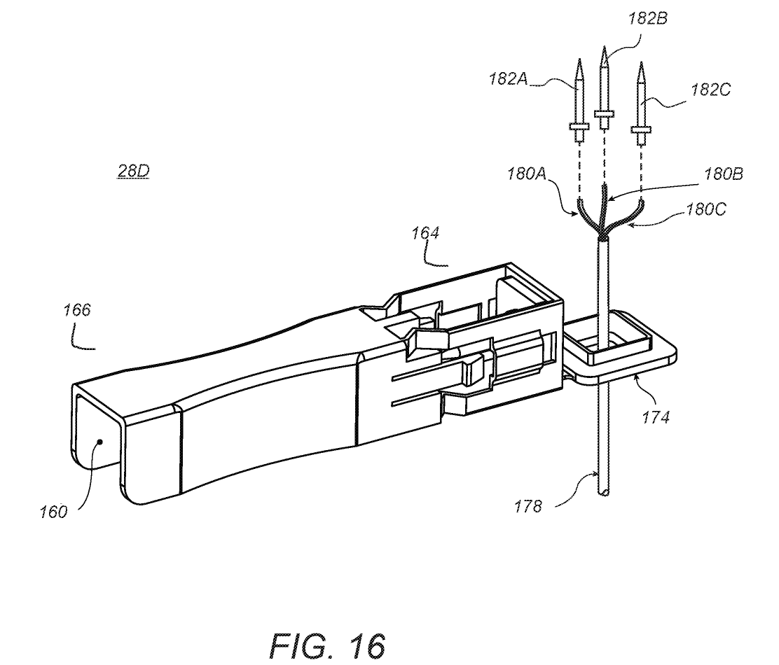

FIG. 16 is the first electrical connector embodiment 28D of FIG. 14 with partial installation of an electrical contact/cable assembly. The electrical contact/cable assembly in the embodiment of FIG. 16 includes a multi-conductor cable 178 having three single conductors 180A, 180B, 180C. In other embodiments, an electrical contact/cable assembly may include one conductor, four or more conductors, or even no conductors. Instead, for example, the electrical contact/cable assembly in some embodiments may be a fixed or flexible mechanical member that provides loss-prevention, guidance, or other features.

In the embodiment of FIG. 16, the multi-conductor cable 178 has three single conductors 180A, 180B, 180C that are each electrical conductors. The electrical conductors may be substantially formed of copper or some other electrical conductor such as gold or silver. The electrical conductors may be stranded, braided, or formed in a different way, and along these lines, the electrical conductors within the multi-conductor cable 178 may be parallel, adjacent, braided, twisted, or in some other way intertwined or not intertwined. An insulating material may be separately formed around each electrical conductor, or a single insulating material may be formed around a plurality of electrical conductors.

The single conductors 180A, 180B, 180C of the multi-conductor cable 178 embodiment in FIG. 16 are each terminated with a corresponding first electrical contact 182A, 182B, 182C. First electrical contacts 182A, 182B, 182C may be solder-connected, crimp-connected, or electromechanically affixed to respective conductors in some other way. The first electrical contacts 182A, 182B, 182C may have any desirable shape (e.g., round, square, hexagonal), length (e.g., 2 mm, 5 mm, 10 mm), diameter (0.2 mm, 0.5 mm, 1 mm), material (e.g., copper, silver, gold), or other feature. The first electrical contacts 182A, 182B, 182C may all be formed alike (e.g., same shape, size, material, and the like), or in other embodiments, one or more of the first electrical contacts 182A, 182B, 182C may be formed differently. In the embodiment of FIG. 16, first electrical contacts 182A, 182B, 182C are formed as “pins” with a pointed alignment feature to be later received by a corresponding second electrical contact 186A, 186B, 186C (FIG. 19). In other embodiments, these or different electrical contacts may be formed as receptacles (e.g., cylinders, barrels, or the like) to receive a corresponding electrical contact.

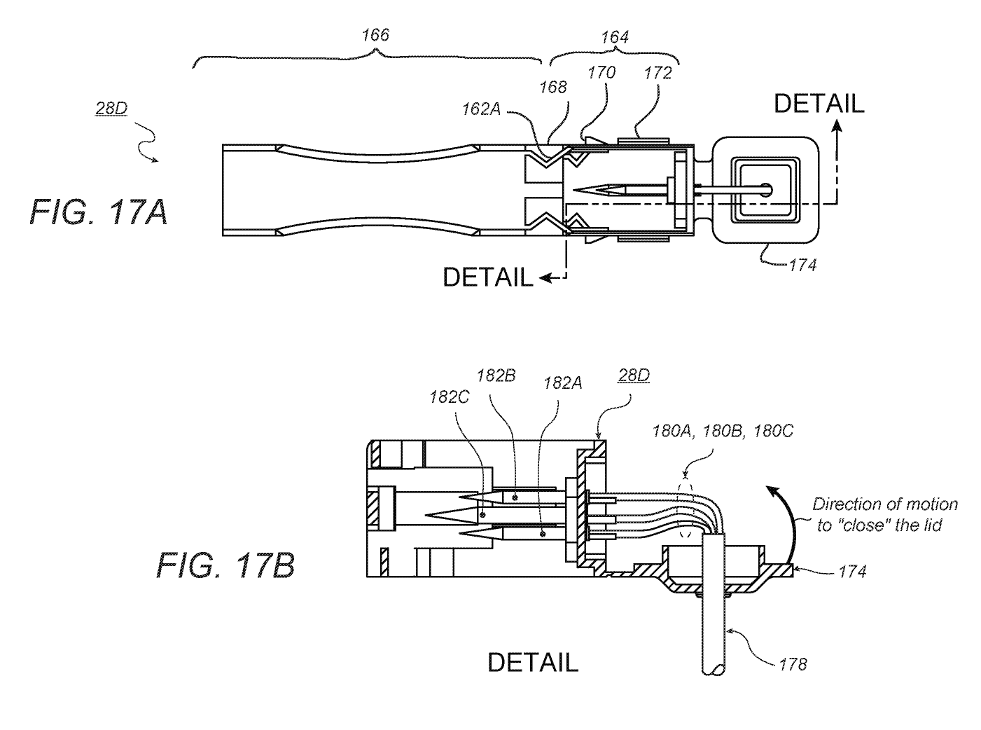

FIG. 17A is a sectional view of the first electrical connector embodiment 28D of FIG. 14 with partial installation of the electrical contact/cable assembly from a top view perspective. FIG. 17B is a detail view of a portion of the first electrical connector embodiment 28D of FIG. 17A from a side view perspective. The partial installation of the electrical contact/cable assembly in FIG. 17A is further along than the partial installation of FIG. 16.

Several features of the first electrical connector embodiment 28D are identified to help orient the structures and their presentation in various ones of FIGS. 14-25C. One live hinge 162A formed between the front and rear portions 166, 164 of the first electrical connector embodiment 28D is identified. A cantilever arm 168 having a locking surface 170 arranged for positive mechanical coupling to a locking receiver 172 is identified.

In the “detail” view of FIG. 17B, the electrical contact/cable assembly has been further advanced through the optional rear lid 174 and into the rear portion 164 of the first electrical connector embodiment 28D. The multi-conductor cable 178 has passed through a hole, cutout, or other aperture of the optional rear lid 174, and the single conductors 180A, 180B, 180C have been advanced toward the front portion of the first electrical connector embodiment 28D. Each one of the first electrical contacts 182A, 182B, 182C has been advanced through a corresponding electrical contact aperture 176 (FIG. 15). In some embodiments, the electrical contact apertures 176 are arranged in a way that provides structural stability and alignment of the first electrical contacts 182A, 182B, 182C.

In some embodiments, the multi-conductor cable 178 is passed through the optional rear lid 174 is also removably or fixedly coupled to the optional rear lid 174. Such coupling can provide structural stability for the first electrical connector embodiment 28D and strain relief for the multi-conductor cable 178. As indicated, in FIG. 17B, when the optional rear lid 174 is “closed” according to the direction indicated, the multi-conductor cable 178 remains in place through the optional rear lid 174, and the single conductors 180A, 180B, 180C are flexibly folded or otherwise arranged within in front of the closed optional lid 174.

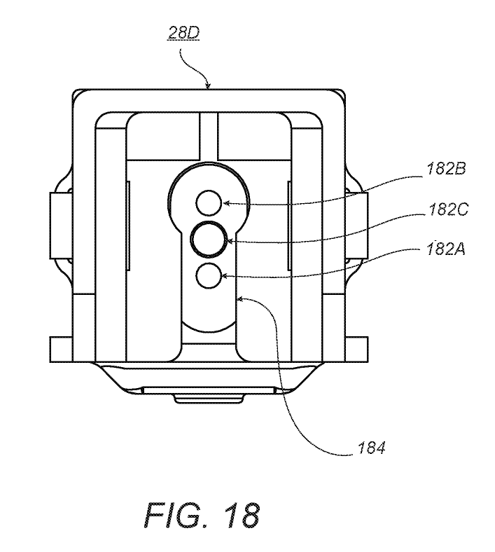

FIG. 18 is a front view of the first electrical connector embodiment 28D of FIG. 14. From the front, an alignment of first electrical contacts 182A, 182B, 182C is evident. The first electrical contacts 182A, 182B, 182C in FIG. 18 are illustrated in a uniform pattern, though other different pattern or non-pattern configurations are contemplated. In FIG. 18, first electrical contact 182C has a different size than first electrical contacts 182A, 182B, and in other embodiments, electrical contacts may have same or different features.

The first electrical connector embodiment 28D in FIG. 18 also shows a mechanical alignment feature 184 integrated therein. The mechanical alignment feature 184 is arranged to facilitate guidance of the first electrical connector embodiment 28D toward a suitable second electrical connector embodiment 36D (FIG. 19) or vice versa.

FIG. 19 is a second electrical connector embodiment 36D beneath, within, or otherwise in a determined proximity to an exemplary contamination barrier 30. With respect to FIG. 1, the second electrical connector embodiment 36D of FIG. 19 might be along the lines of the second electrical connector portion 36 that is placed in the second space 34 above the patient 22 and below the contamination barrier 30. In FIG. 1, the second electrical connector portion 36 is arranged with electrical connector pin receivers 46, and in FIG. 19, the second electrical connector embodiment 36D is arranged with second electrical contacts 186A, 186B, 186C. The second electrical contacts 186A, 186B, 186C of FIG. 19 may be arranged to electromechanically mate with the first electrical contacts 182A, 182B, 182C shown in FIG. 18, respectively.

In one or more alternative embodiments, the second electrical connector embodiment 36D is placed under a contamination barrier 30. The second electrical connector embodiment 36D may be, for example, placed directly on or in proximity to a patient's body, or the second electrical connector embodiment 36D may be placed above a first contamination barrier 30 and below a second contamination barrier 30. In at least one case, For example, the second electrical connector embodiment 36D is integrated with a magnetic sensing device such as the magnetic sensing device 24 of FIG. 2.

In such an exemplary case (FIG. 2), a portion of the second electrical connector embodiment 36D (e.g. the housing) becomes part of the magnetic sensing device. The magnetic sensing device gets placed directly on the patient. A sterile contamination barrier 30 is placed over the second electrical connector embodiment 36D and most of the patient. A cut-out or other access means in the contamination barrier is positioned in proximity to where the skin of the patient is going to be pierced. The patient's skin at that location is sanitized. The medical instrument to be guided (e.g., a stylet) can be laid on top of the contamination barrier 30, and the electrical connection is made (e.g., by piercing the contamination barrier 30 and coupling a first electrical connector embodiment 28 to a second electrical connector embodiment 36, which may be coupled to the magnetic sensing device.

Not shown in FIG. 19, the second electrical connector embodiment 36D is arranged to have a trackable structure 24 electrically coupled thereto.

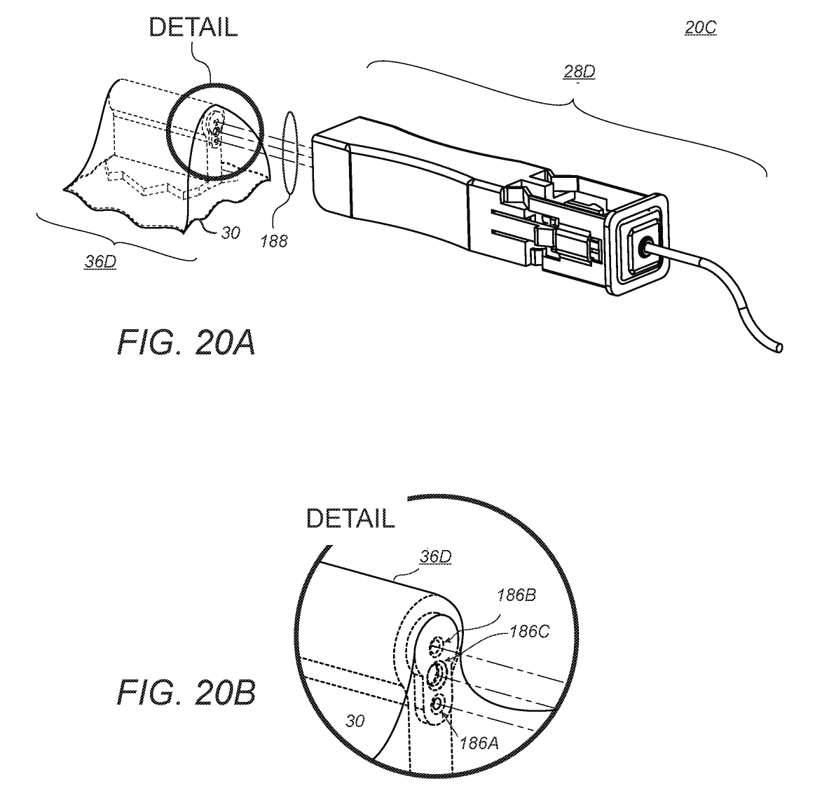

FIGS. 20A and 20B illustrate the electromechanical coupling of the first electrical connector embodiment 28D with the second electrical connector embodiment 36D through a contamination barrier 30. Together, the first and second electrical connector embodiments 28D, 36D form a two-stage electrical connector system 20C. More particularly, FIG. 20A is a two-stage connector housing embodiment and a two-stage connector receiver embodiment aligned for electromechanical coupling through a contamination barrier 30, and FIG. 20B is a detail view of a portion of the two-stage connector housing and two-stage connector receiver aligned for electromechanical coupling through the contamination barrier 30.

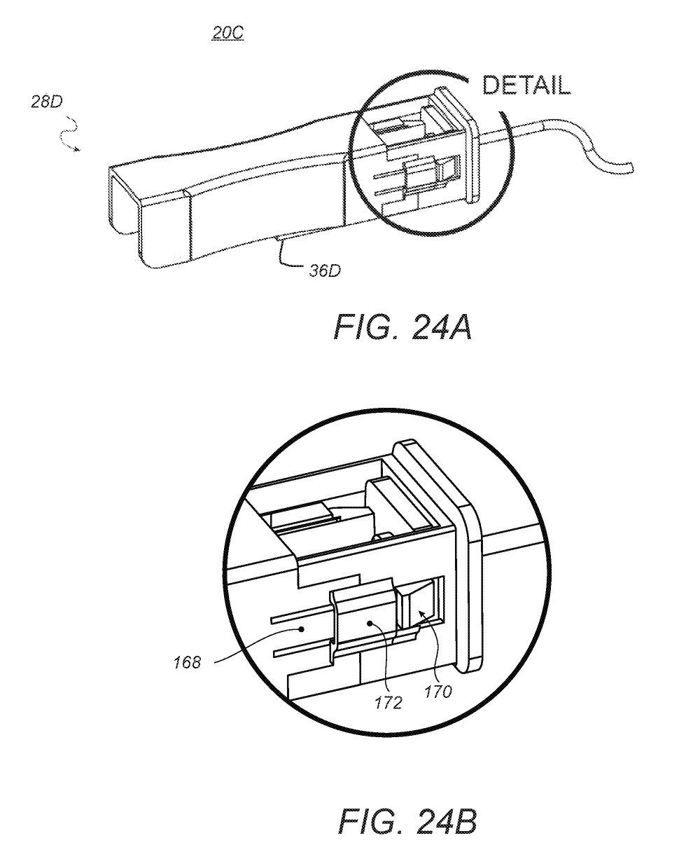

Prior to advancing one or both of the first and second electrical connector embodiments 28D, 36D toward one another along a connection path for first and second electrical contacts 188, it is shown in FIG. 20A that the positive locking mechanism of the first electrical connector embodiment 28D has not yet been engaged. The positive locking mechanism is shown in more detail in FIGS. 24A-24B.

FIGS. 21A and 21B illustrate the electromechanical coupling of the first electrical connector embodiment 28D with the second electrical connector embodiment 36D directly and not through any type of contamination barrier. As in FIGS. 20A and 20B, the first and second electrical connector embodiments 28D, 36D form a two-stage electrical connector system 20C. The two-stage connector system may be formed to work with or without a contamination barrier 30. FIG. 21A is a two-stage connector housing embodiment and a two-stage connector receiver embodiment aligned for direct electromechanical coupling, and FIG. 20B is a detail view of a portion of the two-stage connector housing and two-stage connector receiver aligned for direct electromechanical coupling.

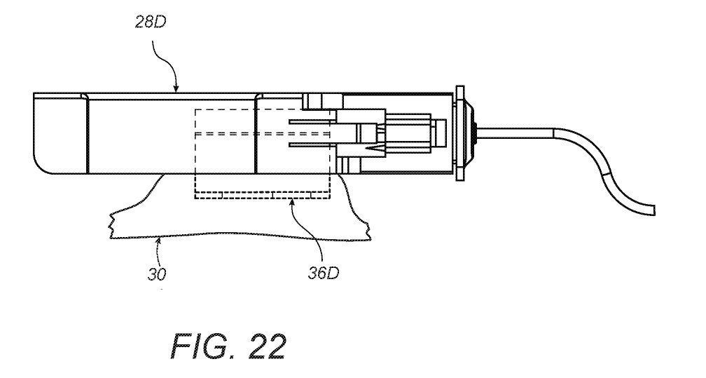

FIG. 22 is a sectional view of the two-stage connector housing and two-stage connector receiver coupled through a contamination barrier. Here, the first electrical connector embodiment 28D, which is above a contamination barrier 30 had been electromechanically coupled to the second electrical connector embodiment 36D, which is below the contamination barrier 30.

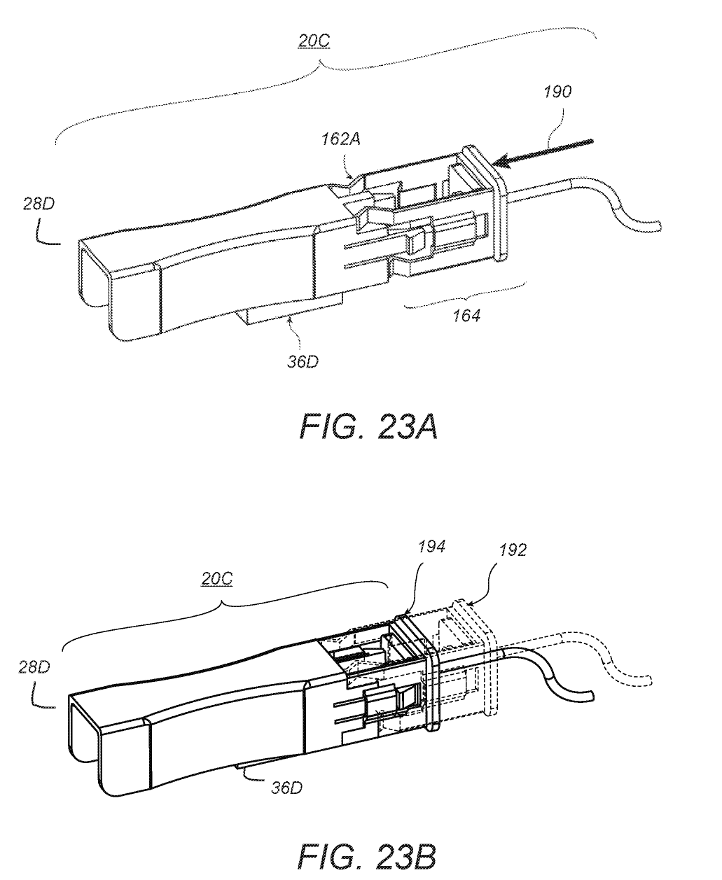

FIGS. 23A and 23B show a button-lock operation of two-stage electrical connector system 20C. Here, FIG. 23A shows a two-stage connector housing embodiment in an open position. FIG. 23B shows the two-stage connector housing embodiment of FIG. 23A advanced to a closed position. In FIG. 23A, a first electrical connector embodiment 28D is aligned above, on, or otherwise in cooperation with a second electrical connector embodiment 36D. In some cases, this first alignment in FIG. 23A includes one or more acts to place the first electrical connector embodiment 28D in proximity to the second electrical connector embodiment 36D with a contamination barrier 30 between the first and second electrical connector embodiments 36D, 28D. In some of these cases, the first and second electrical connector embodiments 36D, 28D may be positively aligned and mechanically coupled by “squeezing” the two connectors together. A haptic or audio feedback may indicate that the first electrical connector embodiment 28D has been mechanically joined to the second electrical connector embodiment 36D.

In at least some of these cases, the one or more acts that mechanically couple the first electrical connector embodiment 28D to the second electrical connector embodiment 36D also arrange a portion of the contamination barrier 30 in a position normal to the direction of travel of the barrier-piercing electrical contacts. For example, Considering the embodiment of FIG. 19, for example, a contamination barrier 30 is arranged over a second electrical connector embodiment 36D wherein the contamination barrier 30 is also “over” the second electrical contacts 186A, 186B, 186C. Returning to FIG. 23A, the mechanical coupling of the first electrical connector embodiment 28D to the second electrical connector embodiment 36d aligns the contamination barrier 30 in a position normal to both the first electrical contacts 182A, 182B, 182C and corresponding second electrical contacts 186A, 186B, 186C. After a first stage including the mechanical coupling, a second stage will electrically and electromechanically couple the electrical contacts of one electrical connector embodiment to another (FIG. 23B).

In at least one case, the force to perform the mechanical coupling of the first electrical connector embodiment 28D to the second electrical connector embodiment 36d is greater than, or greater than or equal to, the force to perform the electrical coupling of the first electrical connector embodiment 28D to the second electrical connector embodiment 36d. In a least one other case, the opposite is true, which means that the force to perform the mechanical coupling of the first electrical connector embodiment 28D to the second electrical connector embodiment 36d is less than (or less than or equal to) the force to perform the electrical coupling of the first electrical connector embodiment 28D to the second electrical connector embodiment 36d.

For reference to other exemplary representations of the first electrical connector embodiment 28D in the present disclosure, a first live hinge 162A and the rear portion of the first electrical connector embodiment 28D are identified. Here, the rear portion of the first electrical connector embodiment 28D is configured to operate as a “button.” The direction of advancement of rear portion 164, which may be considered the direction of advancement of the button 190, is shown in FIG. 23A. The live hinges in these embodiments may reduce the amount of force necessary to engage the positive locking mechanism that indicates successful advancement of the button.

In the two-stage electrical connector system 20C of FIG. 23B, the button in the open position 192 is shown in dashed line as a first starting position. In the first starting position, the first electrical connector embodiment 28D is aligned with the second electrical connector embodiment 36D, but there is no electrical connection between the first and second electrical connector embodiments 28D, 36D. When the button is advanced to the closed position 194, which is illustrated in solid line, the positive locking mechanism (FIG. 24) has been engaged, and there is a robust electrical connection between the first and second electrical connector embodiments 28D, 36D

FIGS. 24A and 24B show the depressed button of two-stage electrical connector system 20C. FIG. 24A is a two-stage connector housing embodiment in a closed and locked position, and FIG. 24B is a detail view of the portion of the two-stage connector housing. Considering the detail illustrated in FIG. 24B, after the button, which is the rear portion 164 of the first electrical connector embodiment 28D, has been depressed, the positive locking mechanism formed in at least some embodiments with live hinges, cantilever arms, locking surfaces, and locking surfaces, is engaged. In the detail view, a first cantilever arm 168 is shown wherein the locking surface 170 has engaged the locking receiver 172. When a user (e.g., a medical practitioner) depresses the button, the user will feel a distinct “click” or other haptic response when the locking mechanism engages. In some cases, when the locking mechanism engages, the user may also hear a distinct “click.” From one or more such responses, the engagement of the locking mechanism indicates to the user that the first electrical connector embodiment 28D has mechanically engaged with the second electrical connector embodiment 36D, and the first and second electrical connector embodiments 28D, 36D are in a robust electrically connected configuration.

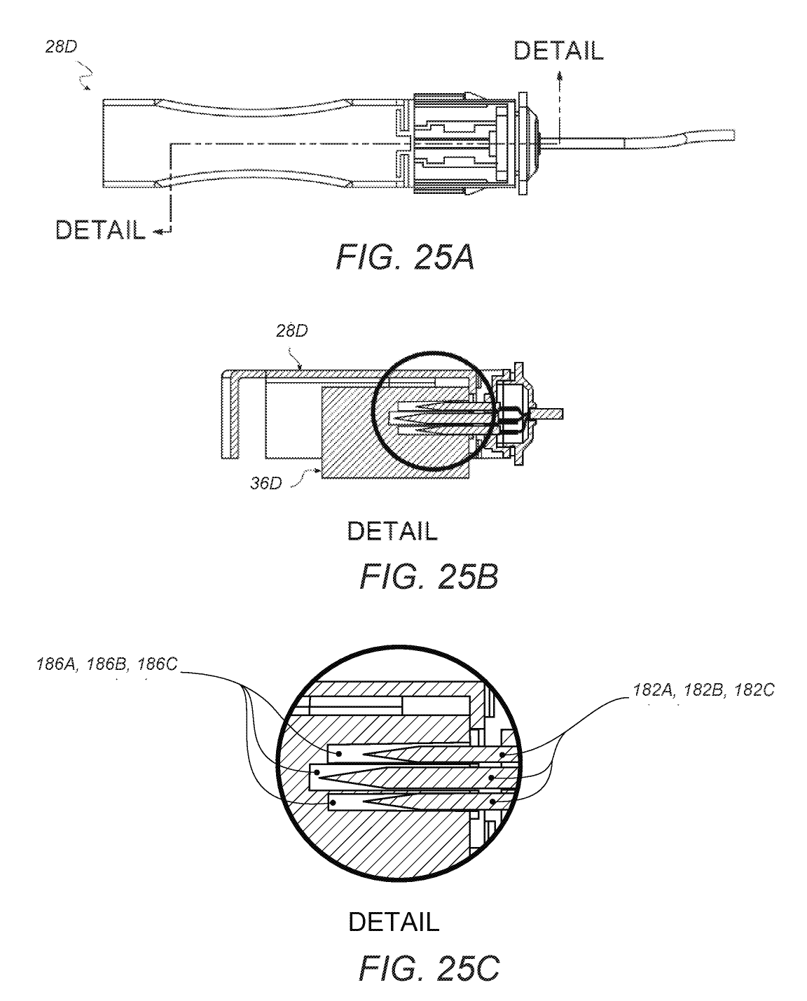

FIGS. 25A, 25B, and 25C show the two-stage electrical connector system 20C in a closed and locked position. FIG. 25A is a sectional view of the two-stage connector housing embodiment from a top view perspective, and FIG. 25B is a detail view of a portion of the two-stage connector housing embodiment of FIG. 25A from a side view perspective. In FIG. 25B, the first electrical connector embodiment 28D and the second electrical connector embodiment 36D are shown in a portion of the two-stage electrical connector system 20C. FIG. 25C is a more detailed view of the portion of the two-stage connector housing embodiment of FIG. 25B. In the detail view, the robust electrical connection of first electrical contacts 182A, 182B, 182C and second electrical contacts 186A, 186B, 186C is shown.

In the embodiment of the two-stage electrical connector system 20C, as shown in FIG. 25C, the first and second electrical contacts are friction fit to provide a clean, low-resistance (e.g., nominally zero ohms) electrical connection. A determined surface area of each first electrical contact is in contact with a determined surface area of each respective electrical contact. The determined surface area of electrical contact may be about 20 to 100 square millimeters (mm²), and other determined surface areas less than 20 mm² and greater than 100 mm² are also contemplated. Beneficially in at least some embodiments, the electrical connection of a first electrical contact to a second electrical contact also provides for an air-gap between a distal end of the first electrical contact and the “bottom” of the corresponding second electrical contact.

In the embodiments described herein, one or more complete or partial embodiments of the electrically conductive path 42 may be formed with one or more wires, conductive shields, conductive cores, meshed wires, braided wires, or some other technique or structure to pass an electrical signal. In some cases, the electrically conductive path 42 may take on another form.

In the embodiments described herein, structures that are coupled together include a direct electrical connection, a remote electrical connection, or some other electrical connection technique. In addition, the coupling may be through one or more intervening devices. The coupling may optionally include a mating or other association of one or more mechanical registration features. In some cases, the coupling may take on another form.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

The various embodiments described above can be combined to provide further embodiments. For example, and without limitation, it is contemplated that any of the electrically conductive structures or electrically insulating structures of one embodiment may be formed using electrically conductive or insulating materials, as the case may be, that are described with respect to any other embodiment. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
a trackable structure having an integrated electromagnet circuit, the trackable structure arranged to controllably produce a magnetic field;
an interface to produce a positional representation of the trackable structure when the trackable structure is within a human body;
a magnetic field sensing device arranged to drive the integrated electromagnet circuit of the trackable structure and arranged to provide position information to the interface; and
a multipart connector to electrically couple the magnetic field sensing device to the trackable structure, the multipart connector including a first connector portion and a second connector portion.

2. A system according to claim 1, wherein the trackable structure includes a medical device and a trackable conductor.

3. A system according to claim 2, wherein the trackable conductor is arranged to receive an electromagnetic drive signal and arranged to generate an electromagnetic field in correspondence with the electromagnetic drive signal.

4. A system according to claim 3, wherein the magnetic field sensing device is arranged to generate position information representing a location of the trackable structure in real time by sensing the electromagnetic field generated by the trackable conductor.

5. A system according to claim 1, wherein the first connector portion and the second connector portion are arranged to form at least one electrically conductive path through the multipart connector when the first connector portion and the second connector portion are mechanically joined together.

6. A system according to claim 5, wherein the magnetic field sensing device is configured to direct passage of an electromagnetic drive signal through the at least one electrically conductive path.

7. A system according to claim 5, wherein the first connector portion includes:
an electrically conductive core having a body, a distal end, and a core electrical contact area formed on the distal end of the electrically conductive core;
a first insulator layer substantially surrounding the body of the electrically conductive core;
a first conductive shield layer substantially surrounding the first insulator layer, the first conductive shield layer having a body, a distal end, and a first electrical contact area formed on the distal end of the first conductive shield layer;
a second insulator layer substantially surrounding the body of the first conductive shield layer;
a second conductive shield layer substantially surrounding the second insulator layer, the second conductive shield layer having a body, a distal end, and a second electrical contact area formed on the distal end of the second conductive shield layer; and
a third insulator layer substantially surrounding the body of the second conductive shield layer.

8. A system according to claim 7, wherein the core electrical contact area, the first electrical contact area, and the second electrical contact area are exposed to an outside environment.

9. A system according to claim 7, wherein the distal end of the electrically conductive core, the distal end of the first conductive shield layer, and the distal end of the second conductive shield layer are configured to pass through a contamination barrier when the first connector portion and the second connector portion are mechanically joined together.

10. A system according to claim 7, wherein the second connector includes:
an electrically conductive conduit having a body, a distal end, and an electrical receiver arranged to receive the core electrical contact area of the first connector portion;
a third insulator layer substantially surrounding the body of the electrically conductive conduit;
a third conductive shield layer substantially surrounding the third insulator layer, the third conductive shield layer having a body, a distal end, and a third electrical receiver formed at the distal end of the third conductive shield layer, the third electrical receiver arranged to receive the first electrical contact area;
a fourth insulator layer substantially surrounding the body of the third conductive shield layer;
a fourth conductive shield layer substantially surrounding the fourth insulator layer, the fourth conductive shield layer having a body, a distal end, and a fourth electrical receiver formed at the distal end of the fourth conductive shield layer, the fourth electrical receiver arranged to receive the second electrical contact area; and
a fifth insulator layer substantially surrounding the body of the fourth conductive shield layer.

11. A system according to claim 7, wherein the first connector portion includes:
a shroud arranged to at least partially enclose the core electrical contact area, the first electrical contact area, and the second electrical contact area.

12. A method, comprising:
providing a contamination barrier to separate a first space from a second space;
providing a first connector portion of a multipart connector in the first space, wherein the first connector portion is arranged for coupling to a magnetic field sensing device;
providing a second connector portion of the multipart connector in the second space, wherein the second connector portion is arranged for coupling to a trackable structure having an integrated electromagnet circuit, the trackable structure arranged to controllably produce a magnetic field;
passing at least one electrical conductor of the first connector portion through the contamination barrier; and
mechanically coupling the first connector portion to the second connector portion thereby forming at least one electrically conductive path through the contamination barrier.

13. A method according to claim 12, wherein the first space has a first level of sterility and the second space has a second level of sterility, the first level of sterility representing a less sterile condition than the second level of sterility.

14. A method according to claim 12, comprising:
prior to passing the at least one electrical conductor of the first connector portion through the contamination barrier, and prior to mechanically coupling the first connector portion to the second connector portion, coupling the second connector portion to the trackable structure.

15. A method according to claim 12, comprising:
applying an electromagnetic drive signal to the integrated electromagnet circuit via the at least one electrically conductive path.

16. A method according to claim 15, wherein applying the electromagnetic drive signal, further comprises:
passing an alternating current excitation signal through the at least one electrically conductive path to the integrated electromagnet circuit of the trackable structure, the alternating current excitation signal having a frequency below 10,000 Hz.

17. A method to form a first electrical connector, comprising:
providing an electrically conductive core having a distal end and a body;
forming a first insulator layer substantially surrounding the body of the electrically conductive core, the first insulator layer formed substantially coaxial with the electrically conductive core;
exposing a core electrical contact area on the distal end of the electrically conductive core;
forming a first conductive shield layer substantially surrounding the first insulator layer, the first conductive shield layer having a body and a distal end, the first conductive shield layer formed substantially coaxial with the first insulator layer;
forming a second insulator layer substantially surrounding the first conductive shield layer, the second insulator layer formed substantially coaxial with the first conductive shield layer;
exposing a first electrical contact area on the distal end of the first conductive shield layer;
forming a second conductive shield layer substantially surrounding the second insulator layer, the second conductive shield layer having a body and a distal end, the second conductive shield layer formed substantially coaxial with the second insulator layer;
forming a third insulator layer substantially surrounding the second conductive shield layer, the third insulator layer formed substantially coaxial with the second conductive shield layer; and
exposing a second electrical contact area on the distal end of the second conductive shield layer.

18. A method to form a first electrical connector according to claim 17, wherein the distal end of the electrically conductive core is arranged to pierce a contamination barrier.

19. A method to form a second electrical connector, comprising:
providing an electrically conductive multi-leaf receiver having a first electrical receiver end and a body;
forming a first insulator layer substantially surrounding the body of the electrically conductive multi-leaf receiver, the first insulator layer formed substantially coaxial with the electrically conductive multi-leaf receiver;
forming a first electrically conductive receiver substantially surrounding the first insulator layer, the first electrically conductive receiver having a second electrical receiver end and a body, the first electrically conductive receiver substantially coaxial with the first insulator layer;
forming a second insulator layer substantially surrounding the body of the first electrically conductive receiver, the second insulator layer formed substantially coaxial with the first electrically conductive receiver;
forming a second electrically conductive receiver substantially surrounding the second insulator layer, the second electrically conductive receiver having a second electrical receiver end and a body, the second electrically conductive receiver substantially coaxial with the second insulator layer; and
forming a third insulator layer substantially surrounding the body of the second electrically conductive receiver, the third insulator layer formed substantially coaxial with the second electrically conductive receiver.

20. A method to form a second electrical connector according to claim 19, wherein the bodies of the insulator layers and the electrically conductive receivers are flexible.

21. A first electrical connector, comprising:
a group of electrical connector pins arranged to pass through a contamination barrier and pass an electromagnetic drive signal, each electrical connector pin has a distal end;
an electrically conductive path coupled to the group of electrical connector pins, the electrically conductive path is arranged to pass the electromagnetic drive signal;
an electrical housing that contains the electrical connector pins and the electrically conductive path; and
an insulating material inside the electrical housing, the insulating material holds the group of electrical connector pins and the electrically conductive path in place.

22. A first electrical connector according to claim 21, wherein the distal ends of the group of electrical connector pins are arranged to pass through a contamination barrier.

23. A second electrical connector, comprising:
a group of electrical pin receivers arranged to receive a first electrical connector and pass an electromagnetic drive signal, each electrical pin receiver has an electrical receiver end;
an electrically conductive path coupled to the group of electrical pin receivers, the electrically conductive path is arranged to pass the electromagnetic drive signal;
an electrical housing that contains the electrical pin receivers and the electrically conductive path; and
an insulating material located inside the electrical housing, the insulating material holds the electrical pin receivers and the electrically conductive path in place.

24. A second electrical connector according to claim 23, the electrical receiver ends of the group of electrical pin receivers are arranged to receive a group of electrical connector pins.
